(19) United States
(12) Patent Application Publication
(10) Pub. No.: US 2012/0237563 A1
(43) Pub. Date: Sep. 20, 2012

(54) CONTROLLED-RELEASE FORMULATIONS OF ANABASEINE COMPOUNDS AND USES THEREOF

(75) Inventors: William Reade Kem, Gainesville, FL (US); Ferenc Soti, Gainesville, FL (US); Robert Freedman, Englewood, CO (US); Lynn Johnson, Morrison, CO (US)

(73) Assignee: UNIVERSITY OF FLORIDA RESEARCH FOUNDATION INC., Gainesville, FL (US)

(21) Appl. No.: 13/391,371

(22) PCT Filed: Aug. 18, 2010
(86) PCT No.: PCT/US10/45867

§ 371 Date: May 31, 2012

Related U.S. Application Data
(60) Provisional application No. 61/235,876, filed on Aug. 21, 2009.

(51) Int. Cl. A61K 31/444; A61P 25/18; A61K 9/00; A61P 25/00; A61P 29/00; A61P 35/00; A61P 25/28; A61P 25/16

(52) U.S. Cl. 424/400; 514/334

(57) ABSTRACT

The subject invention pertains to controlled-release dosage forms of anabaseine compounds, such as 3-(2,4-dimethoxybenzylidene)-anabaseine (also known as DMXBA or GTS-21) or a pharmaceutically acceptable salt thereof, methods of use, and methods for producing controlled-release dosage forms.

### CROSS-REFERENCE TO RELATED APPLICATION

The present application claims the benefit of U.S. Application Ser. No. 61/235,876, filed Aug. 21, 2009, which is hereby incorporated by reference herein in its entirety, including any figures, tables, nucleic acid sequences, amino acid sequences, and drawings.

### GOVERNMENT SUPPORT

This invention was made with government support under National Institute of Medical Health grant number R01 MH061412. The government has certain rights in the invention.

### BACKGROUND OF THE INVENTION

Several types of nicotinic acetylcholine receptors (nAChRs) are known to play a role in central nervous system activity and as such are involved in cognition, mood, and neuroprotection. The various types of known nicotinic ligands appear to have different combinations of effects on nicotine-modulated functions, depending on the subtypes of nAChRs affected, some affecting all receptors, others having more selective actions.

Acetylcholine receptors can be divided into muscarinic (mAChR) and nicotinic (nAChR) subtypes in the mammalian central nervous system (CNS). These subtypes are distinguished based on their ability to be stimulated by either the mushroom toxin muscarine or the plant alkaloid nicotine. Nicotinic receptors are important in cholinergic transmission in autonomic ganglia, striated muscles, the neuromuscular junction, and in brain and spinal synapses. Some nAChRs are also expressed in non-neuronal or muscle cells. Within the nervous system, these non-neuronal cells include microglia and astrocytes; outside the nervous system, non-neuronal cells expressing alpha7 receptors include macrophages, vascular endothelium and pulmonary epithelial cells.

All known mammalian nAChRs are cation selective ligand-gated ion channels that form pentameric structures in the plasma membrane. Each subunit of the pentamer contains four transmembrane domains. There are at least seventeen different nAChR subunit genes, including five found in striated muscle (alpha1, beta1, gamma, delta, sigma) and twelve neuronal nAChR subunits (alpha2-10, beta2-4). These channels can be composed of a number of different combinations of subunits. Examples of the most abundant subtypes in the brain include the a7 subtype (a-bungarotoxin-sensitive) and the alpha4beta2 subtypes (alpha4(2)beta2(3) or alpha4(3)beta2(2)). There is strong evidence supporting the notion that most alpha7 receptors are expressed as homopentamers. Functional bungarotoxin-sensitive channels are expressed in Xenopus oocytes when only alpha7 cDNA is injected. However, rat hippocampal interneurons also have alpha7-containing nAChRs that exhibit pharmacological and functional properties different from those of homomeric alpha7 receptors. The co-expression of the alpha7 subunit with the beta2 subunit in Xenopus oocytes has produced functional heteromeric channels with similar properties to the rat hippocampal interneuron alpha7-containing receptor (Khiroug et al., 2004, J Physiol. (London), 540:425-434). In addition to its ability to assemble into homomeric channels, the alpha7 nAChR channel displays much greater permeability to calcium ions than other nAChRs or the NMDA glutamate receptor subtype.

The nAChRs in the brain have long been recognized as being important in mediating the euphoric effects of nicotine. Neuronal nAChR deficits have also been implicated in several diseases including Alzheimer's disease (AD) and schizophrenia. Until recently, the study of neurodegenerative diseases focused on the muscarinic type neuronal acetylcholine receptor (mAChR) because of its abundance in the brain when compared to the population of neuronal nicotinic receptors (nAChRs). However, the discovery of a greater relative loss of nicotinic receptors than of muscarinic receptors in the brains of Alzheimer's patients, as well as evidence that nicotinic agonists enhance cognition, has spurred interest in nAChRs (Sabbagh et al., 1998, J. Neural Transm., 105:709-717). The two major brain nAChRs, alpha4beta2 and alpha7, are important for cognitive processes such as attention, learning, and memory. Since brain alpha7 nicotinic receptors are spared relative to the alpha4beta2 nAChRs in AD and also possess exceptionally high calcium ion permeability, they are considered a particularly promising therapeutic target for treatment of AD. In addition to their direct involvement in synaptic transmission, certain nicotinic receptor subtypes, particularly alpha7, because of their very high calcium permeability, also stimulate calcium-dependent intracellular signal transduction processes that are neuroprotective by maintaining neuronal integrity in the presence of stressful states such as ischemia or mechanical trauma.

The characteristic pathology of AD includes extracellular beta-amyloid plaques, intracellular neurofibrillary tangles, loss of neuronal synapse and pyramidal cells. The cholinergic dysfunction in AD is represented by a reduction in the activity of the ACh-synthesizing enzyme cholineactyltransferase (ChAT) and a loss of neuronal and glial nAChRs. This alteration is possibly attributable to a preferential loss of cholinergic nerve terminals. In schizophrenia, there is a disruption in the normal brain mechanism that eliminates repetitive stimuli in order to avoid the distraction of such stimuli and enhance concentration upon the mental task at hand. This malfunction in the simple filter for sensory input causes an overload of stimuli, which may lead to misperceptions of sensory stimuli producing delusions, or withdrawal from stimuli, causing schizoid behavior. It has been shown that the schizophrenic hippocampus, a key part of the brain for cognitive function, displays reduced expression of alpha7-type nAChRs, which have been shown to mediate sensory gating.

The alpha7 nAChR subtype has been reported to play a role in several diseases of the central nervous system (CNS) including Alzheimer's disease (Wang et al., 2000, J Biol. Chem., 275(8):5626-32; Kem, 2000, Brain Biol. Res., 113(1-2):169-81), schizophrenia (Adler et al., 1998, Schizophr Bull, 24(2):189-202), Parkinson's disease (Quik et al., 2000, Eur. J. Pharm. 393(1-3):223-2) and attention deficit-hyperactivity disorder (Wilens et al., 1999, Am. J. Psychiatry, 156(12):1931-1937; Levin et al., 2000, Eur. J. Pharmacol., 393(1-3):141-146). Many agents have been identified that act as selective agonists of the alpha7 nAChR subtype and have been proposed as useful for the treatment of these and other central nervous system conditions (de Fiebre et al., 1995, Mol. Pharmacol., 47:164-171; Kem et al., 2000, Behav. Brain Res., 13(1-2):169-81; Kem et al., 2004, Mol. Pharmacol. 65:56-67; Papke et al., 2009, JPET, 329:1-17; Horenstein et al., 2008, Mol. Pharmacol., 74:1496-1511; Papke et al., 2004, Neuropharmacology, 46:1023-1038; U.S. Pat. Nos. 6,110,914; 5,902,814; 6,599,916; and 6,432,975).

It is now known that selective alpha7 nicotinic receptor agonists can improve memory-related behaviors and protect against neurotoxicity induced by trophic factor deprivation, amyloid exposure, excitotoxicity, in vivo ischemia and axotomy. The alpha7 nAChR subtype is known to cause long-term synaptic modulation through its influence on glutamatergic synapses. Strong, brief stimulation of presynaptic alpha7-containing nAChRs can enhance hippocampal glutamatergic synaptic transmission for some time after the nicotinic agonist has been removed. Alpha7 receptor agonists enhance cognition and auditory-gating processes and thus are attractive drug candidates for the treatment of senile dementias and schizophrenia (Freedman et al., 1996, Arch. Gen. Psychiatry, 53:1114-1121; Stevens et al., 1998, Psychopharmacology (Berl), 136:320-327; Kem, 2000, J. Pharmacol. Exp. Ther., 283:979-992).

Anabaseine (2-(3-pyridyl)-3,4,5,6-tetrahydropyridine) is a naturally occurring alkaloid toxin produced by some marine worms, which use the substance to paralyze prey and deter predators (Kem, et al., 1971, Toxicon, 9:23-32). Anabaseine is structurally related to nicotine, differing from the tobacco alkaloid in having a tetrahydropyridyl ring rather than a pyrrolidinyl ring. Like nicotine, anabaseine stimulates all nAChRs (Kem et al., 1997, J. Pharmacol. Exp. Ther. 283:979-992). It acts as an agonist at central and peripheral nicotinic receptors (de Fiebre et al., 1995, Mol. Pharmacol., 47:164-171; Kem et al., 1997, J. Pharmacol. Exp. Ther., 283:979-992). A synthetic disubstituted benzylidene derivative of anabaseine, 3-(2,4-dimethoxybenzylidene)-anabaseine (also known as DMXBA, DMXB, and GTS-21) is a compound that, at relatively low concentrations, selectively stimulates human alpha7 subunit-containing nAChRs, and is an antagonist at alpha4beta2 receptors at higher concentrations (Briggs et al., 1995, Neuropharmacology, 34:583-590; Kem et al., 2000, Behav. Brain Res., 113:169-181). DMXBA enhances cognitive behavior in aging and nucleus basalis-lesioned mammals, including monkeys, and is currently in clinical trials to determine whether it can ameliorate cognitive dysfunction associated with Alzheimer's disease and schizophrenia (Woodruff-Pak et al., 1994, Brain Res., 645:309-317; Arendash et al., 1995, Brain Res., 674:252-259; Briggs et al., 1997, Pharmacol. Biochem. Behav., 57:231-241; Buccafusco and Terry, 2000, J. Pharmacol. Exp. Ther., 295:438-446; Kitagawa et al., 2003, Neuropsychopharmacology 28:542-551; Freedman et al., 2008, Am. J. Psychiatry, 165:1040-1047). Benzylidene-anabaseines (example: DMXBA) and cinnamylidene-anabaseines (example: DMAC-anabaseine, Meyer et al., 1998) also display neuroprotective properties against the neurotoxic actions of β-amyloid (Martin et al., 1994, Drug Dev. Res., 31:134-141; Kihara et al., 1997, Ann. Neurol., 42:159-163; Shimohoma et al., 1998, Brain Res., 779:359-363) and in animal models of stroke. Additionally, DMXBA has been demonstrated to inhibit the self-administration of nicotine by rats (U.S. Pat. No. 5,977,144, Meyer et al., to the University of Florida; International Publication No. WO/1999/010338, Meyer et al., to the University of Florida).

DMXBA is less toxic than nicotine and does not affect autonomic and skeletal muscle systems at doses used to enhance cognitive behavior. Initial pharmacokinetic analyses indicated that DMXBA is rapidly metabolized after oral administration (Mahnir et al., 1998, Biopharm. Drug Dis., 19:147-151; Azuma et al., 1999, Xenobiotica, 29(7):747-762). Clinical studies of DMXBA indicate that large doses can be safely administered orally without adverse effects and that the drug has a half-life of about two hours or less (Kitagawa et al. 2003; Hashimoto et al., 2005, Curr. Med. Chem. —Central Nervous System Agents, 5:171-184; Olincy et al., 2006, Arch. Gen. Psychiat., 63:630-638). It rapidly enters the brain after oral administration and enhances cognitive behavior (Kem et al., 2004, Molecular Pharmacology, 65(1):56-67). The pharmacokinetic profile of DMXBA is not optimal, however. Although it is rapidly absorbed and distributed into the brain, it has a relatively modest bioavailability. Because the half-life of DMXBA is relatively short, it often requires frequent administration, which makes it less practical for use in a cognitively impaired, non-adherent patient population (Olinsky and Stevens, 2007, Biochem. Pharmacol., 74(8):1192-1201). Hydroxy metabolites of DMXBA are more potent than DMXBA on human alpha7 receptors; however, they are rapidly extracted from circulation by the liver and they do not readily enter the brain (Kem et al., 2004).

The importance of developing highly potent and selective alpha7 nicotinic receptor agonists has increased as the role of these receptors in cognitive dysfunction and degenerative disease becomes clearer. The cinnamylidene-anabaseines have similar pharmacological as well as chemical properties to the benzylidene derivatives, and some are even more potent alpha7 agonists than DMXBA (U.S. Pat. No. 5,977,144, Meyer et al., to the University of Florida; International Publication No. WO/1999/010338, Meyer et al., to the University of Florida). Efforts are also being made to design new anabaseine analogs possessing greater bioavailability and brain penetration properties.

### BRIEF SUMMARY OF THE INVENTION

The present invention provides controlled-release formulations (dosage foiins) of anabaseine compounds, such as 3-(2,4-dimethoxybenzylidene)-anabaseine (also known as DMXBA, DMXB, and GTS-21), and methods for the treatment or prevention of various diseases or conditions caused or exacerbated by defects in, and/or malfunctioning of central nervous system nicotinic acetylcholine receptors (nAChRs), such as Alzheimer's disease, schizophrenia, Parkinson's disease, and attention deficit-hyperactivity disorder; peripheral nAChRs; or non-neuronal nAChRs, such as inflammatory conditions (e.g., rheumatoid arthritis), trauma, hemorrhage, deficient angiogenesis, excessive angiogenesis, or abnormal cell proliferation (e.g., cancer). In these methods, the disease or condition in a subject is treated or prevented by administering an effective amount of one or more controlled-release formulations of anabaseine compounds to produce desired plasma levels and/or brain levels of the compound(s) or their active metabolite(s) in the subject. Also provided are methods for providing desired plasma levels and/or brain levels of anabaseine compounds or their active metabolites in a subject. The anabaseine compound may be an anabaseine agonist or an anabaseine antagonist.

The defective and/or malfunctioning nicotinic acetylcholine receptor type(s) may be, for example, a neuronal nicotinic receptor or a non-neuronal nicotinic receptor, or both types may be defective and/or malfunctioning. In some embodiments, the nicotinic acetylcholine receptors are neuronal nicotinic acetylcholine receptors (e.g., brain nicotinic receptors such as alpha7-containing receptors, alpha4(2)beta2(3), or alpha4(3)beta2(2)), and the disease or condition is Alzheimer's disease, schizophrenia, Parkinson's disease, attention deficit-hyperactivity disorder, or another disease or condition caused or exacerbated by a defect in, and/or malfunctioning of nAChRs. In some embodiments, the nicotinic acetylcholine receptors are non-neuronal nicotinic acetylcholine receptors, and the disease or condition is an inflammatory disorder (such as rheumatoid arthritis), trauma, hemorrhage, deficient angiogenesis, excessive angiogenesis, abnormal cell proliferation (e.g., cancer), or another disease or condition caused or exacerbated by a defect in, and/or malfunctioning of, a non-neuronal nAChR (which may also be of the alpha7 sub-type).

The present invention is also directed to controlled-release formulations, including, for example, tablet and capsule formulations suitable for oral administration, of anabaseine compounds such as DMXBA. These formulations are useful in the treatment or prevention of various diseases or conditions associated with defects in, and/or malfunctioning of, nicotinic acetylcholine receptors in a mammalian subject, preferably in a human, upon oral administration thereof to the subject. These formulations can also be used in research by administration to an animal model of a disease or condition caused or exacerbated by defects in, and/or malfunctioning of, nicotinic receptors.

The anabaseine compound or compounds may be administered to human or non-human mammalian subjects. For example, the anabaseine compound or compounds may be administered to veterinary patients or animal models of disease. In preferred embodiments of the methods, the subject is a human subject.

Various formulations, routes of administration, and dosing regimens that may be used are described in detail herein. In some embodiments of the methods, the formulation is an intravenous formulation. In some embodiments of the methods, the formulation is an oral formulation. The formulations may include one or more anabaseine compounds together with other optional ingredients. The formulations may be administered in a variety of dosing regimens, including administering one or more formulations that may or may not be administered via the same route of administration. The formulations may also be delivered by repeat dosing and by substantially continuous dosing.

In some embodiments, the one or more anabaseine compounds are prepared with carriers that will protect the one or more anabaseine compounds against rapid elimination from the body, such as a controlled-release matrix, implant, and/or microencapsulated delivery system. In some embodiments, the carrier is a hydrophilic matrix.

This invention provides methods and compositions for oral administration of anabaseine compounds across a range of release rates, including formulations for extended-release of one or more anabaseine compounds (e.g., controlled-release tablets and capsules). In accordance with one aspect of the invention, controlled-release compositions comprising a hydrophilic matrix and one or more anabaseine compounds suitable for oral administration to a subject may be prepared by controlling or varying one or more of the following factors of the composition: drug/polymer ratio (anabaseine compound(s)/polymer ratio), polymer viscosity, polymer hydration characteristics, and active ingredient particle size. Upon ingestion of the controlled-release composition comprising one or more anabaseine compounds and a hydrophilic matrix by a subject, the surface of the controlled-release composition is initially wetted. Specifically, the surface wets and the polymer of the hydrophilic matrix begins to hydrate, forming a gel layer. Any anabaseine compound near the surface of the composition is released. Next, expansion of the gel layer occurs. Specifically, water permeates into the composition, increasing the thickness of the gel layer. Polymer relaxation in the dry core also contributes to dosage swelling. The outer polymer layer becomes fully hydrated, eventually dissolving into the gastric fluids, with water continuing to permeate toward the core of the composition. The one or more anabaseine compounds are released primarily by one of two mechanisms, depending upon their solubility. Soluble anabaseine compounds (along with any other soluble active agents included in the composition) are released primarily by diffusion through the gel layer. Insoluble anabaseine compounds (along with any other insoluble active agents included in the composition) are released primarily through erosion of the composition.

In one aspect of the invention, there is provided a method of treatment comprising orally administering a composition for sustained-release of one or more anabaseine compounds to a subject in need thereof. In another aspect, there is provided a use of a composition suitable for oral administration for sustained-release of one or more anabaseine compounds for treatment of a subject. In another aspect, there is provided a use of a composition suitable for oral administration for extended-release of one or more anabaseine compounds for preparation of a medicament for treatment of a subject. The subject in such cases may, for example, be suffering from Alzheimer's disease, schizophrenia, Parkinson's disease, attention deficit-hyperactivity disorder, or other disease or condition associated with a defect in and/or malfunctioning of brain nicotinic receptors, or other neuronal nicotinic receptors, or non-neuronal nicotinic receptors such as an inflammatory disorder (e.g., rheumatoid arthritis), trauma, hemorrhage, deficient angiogenesis, excessive angiogenesis, abnormal cell proliferation (e.g., cancer).

In another aspect of the invention, there is provided a method for preparing a controlled-release composition, such as an extended-release tablet or capsule, suitable for oral administration, the method comprising: a) blending one or more anabaseine compounds and a matrixing agent, such as a hydrophilic polymer, and, optionally, one or more other ingredients (e.g., lubricant, filler, binder, disintegrant, lubricants, auxiliary excipients such as glidants, solubilizers, etc.), to form a mixture; and b) forming the mixture into the controlled-release composition, e.g., by direct compression, or by wet or dry granulation followed by compression.

In another aspect of the invention, there is provided a composition suitable for oral administration for extended-release of one or more anabaseine compounds, the composition comprising: a) from about 0.1% to about 80% by weight anabaseine compound(s); and from about 99.9% to about 20% by weight extended-release agent. The composition may provide extended-release of the one or more anabaseine compounds over a period of from about 2 hours to about 48 hours. Preferably, the extended-release agent comprises a matrix. In some embodiments, the extended-release agent comprises a hydrophilic polymer, such as cellulose ether. The composition may further include one or more of the following additional ingredients: lubricant, filler, binder, disintegrant, lubricants, auxiliary excipients such as glidants and solubilizers.

In another aspect of the invention, there is provided a method of maintaining a serum anabaseine compound concentration in a subject for a duration of from about 4 hours to about 8 hours, the method comprising administering an effective amount of a controlled-release composition described herein. In another aspect of the invention, there is provided a method of maintaining a concentration of an anabaseine compound in the brain of a subject for a duration of about 4 hours to about 8 hours, the method comprising administering an effective amount of a controlled-release composition described herein.

In another aspect of the invention, there is provided a use of a controlled-release composition for preparation of a medicament for maintaining a serum concentration of an anabaseine compound or active metabolite thereof in a subject for a duration of about 4 hours to about 8 hours. In another aspect of the invention, there is provided a use of a controlled-release composition described herein for preparation of a medicament for maintaining a concentration of an anabaseine compound or active metabolite thereof in the brain of a subject for a duration of about 4 hours to about 8 hours. In another aspect of the invention, there is provided a use of a controlled-release composition described herein for preparation of a medicament for treatment of a disease or condition caused or exacerbated by a defect in, and/or malfunctioning of nAChR.

### BRIEF DESCRIPTION OF THE DRAWINGS

FIG. 1 is a graph showing the release profile of four controlled-release (CR) capsules of DMXBA dihydrochloride in vitro (see Example 1; Table 1).

FIG. 2 is a graph showing the plasma DMXB-A levels in a human subject following administration of the CR formulation and dosage of FIG. 1. FIG. 2 shows that the formulation is producing the sustained plasma levels of DMXB-A predicted from the in vitro tests. After one capsule, potentially therapeutic levels are seen for 6 to 8 hours. Averages of levels from two administrations in the same subject are shown.

FIG. 3 shows the chemical structure of DMXBA (GTS-21) in its free base form.

FIGS. 4A-4C show formula I of U.S. Patent Publication 2005/0288333.

FIG. 5 shows formula II of U.S. Patent Publication 2005/0288333.

FIGS. 6A-6B show a formula for arylidene-anabaseines described in U.S. Pat. No. 5,977,144.

### DETAILED DISCLOSURE OF THE INVENTION

One aspect of the invention is a controlled-release dosage form, comprising a therapeutically effective amount of an anabaseine compound and a controlled-release agent. Preferably, the controlled-release agent is a polymer matrix with the one or more anabaseine compounds incorporated therein. In some embodiments, the controlled-release dosage form is solid dosage form. In some embodiments, the controlled-release dosage form is an oral dosage form. In some embodiments, the controlled-release dosage form is an oral dosage form, such as a tablet, capsule, solution, syrup, elixir, suspension, or powder. In preferred embodiments, the controlled-release dosage form is a solid oral dosage form, such as a tablet or capsule.

Preferably, the polymer of the controlled-release matrix has one or more of the following characteristics: nonionic, water-insoluble, and gel-forming. In some embodiments, the polymer is a gel-forming polymer that hydrates in an aqueous environment, forming a gelatinous (gel) layer on the outer surface of the dosage form, which retards wetting and disintegration of the interior of the dosage form, and the gel layer swells or expands (increases in thickness) as water penetrates into the dosage form and the anabaseine compound diffuses through the gel layer. In cases in which the anabaseine compound is water-insoluble, the compound is preferably released from the dosage form primarily through erosion of the dosage form. In cases in which the anabaseine compound is water-soluble, the compound is preferably released from the dosage form primarily through diffusion through the gel layer.

In some embodiments, the polymer of the controlled-release matrix is a cellulose polymer, such as a cellulose ether. In some embodiments, the polymer is hypromellose or methyl cellulose.

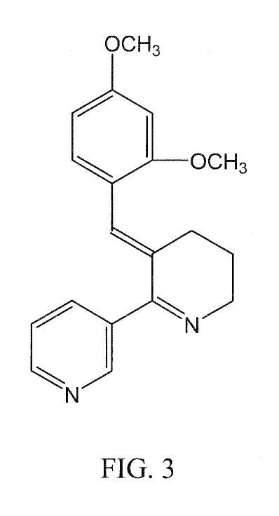

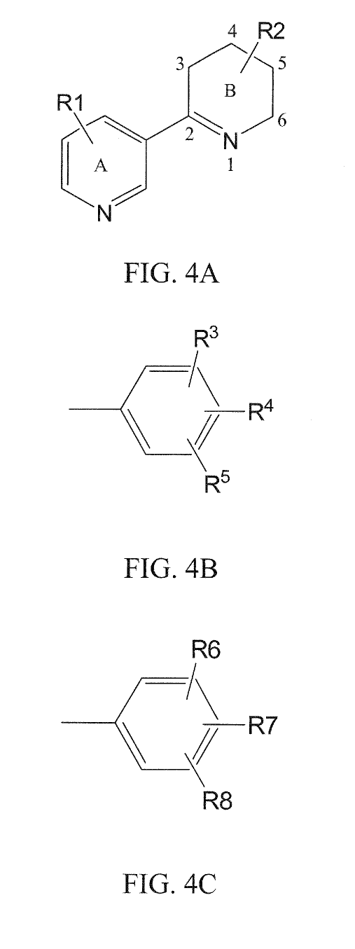

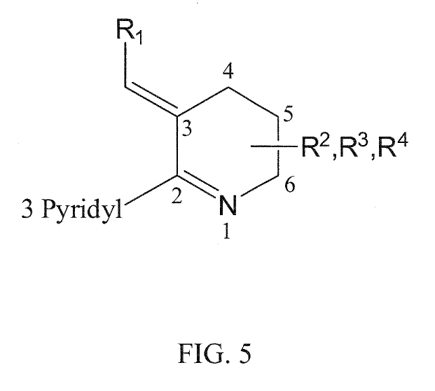

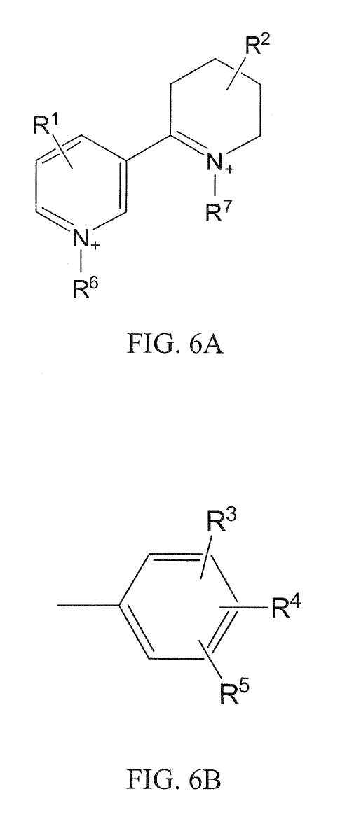

The anabaseine compounds used in the formulations and methods of the invention may have a chemical structure shown in FIGS. 4A-4C, FIG. 5, or FIGS. 6A-6B. In preferred embodiments, the anabaseine compound is an arylidene-anabaseine, such as DMXBA (shown in FIG. 3), or a pharmaceutically acceptable salt thereof.

The anabaseine compounds may be nAChR agonists or antagonists (also referred to in as anabaseine agonists or antagonists). In some embodiments, the anabaseine compound is an agonist of the alpha7 nicotinic acetylcholine receptor, such as DMXBA.

Another aspect of the invention is a method for administering an anabaseine compound to a subject in a controlled-release fashion, comprising administering a controlled-release dosage form described herein to the subject. In some embodiments, a therapeutically effective amount of the anabaseine compound, or an active metabolite thereof, is maintained in the blood and/or brain of the subject for a duration in the range of about 4 hours to about 8 hours. In some embodiments, a therapeutically effective amount of the anabaseine compound, or an active metabolite thereof, is maintained in the blood and/or brain of the subject for a duration in the range of about 6 hours to about 8 hours.

Another aspect of the invention is a method for treating or preventing a disease or condition associated with (caused or exacerbated by) a defect in, and/or malfunctioning of, a nicotinic acetylcholine receptor, the method comprising administering a controlled-release dosage form described herein to a subject in need thereof. In some embodiments, the nicotinic acetylcholine receptor is a neuronal nicotinic acetylcholine receptor of the brain, and the disease or disorder is Alzheimer's disease, schizophrenia, Parkinson's disease, or attention deficit-hyperactivity disorder. In some embodiments, the nicotinic acetylcholine receptor is a non-neuronal nicotinic receptor, and the disease or disorder is an inflammatory disorder (e.g., rheumatoid arthritis), trauma, hemorrhage, deficient angiogenesis, excessive angiogenesis, or abnormal cell proliferation (e.g., cancer). In some embodiments, a therapeutically effective amount of the anabaseine compound, or an active metabolite thereof, is maintained in the blood and/or brain of the subject for a duration in the range of about 4 hours to about 8 hours. In some embodiments, a therapeutically effective amount of the anabaseine compound, or an active metabolite thereof, is maintained in the blood and/or brain of the subject for a duration in the range of about 6 hours to about 8 hours.

Another aspect of the invention is a method for preparing a controlled-release dosage form described herein, suitable for oral administration, the method comprising: a) blending one or more anabaseine compounds and a matrixing agent, and optionally, one or more other ingredients to form a mixture; and b) forming the mixture into the controlled-release dosage form. In some embodiments, the one or more other ingredients includes at least one ingredient selected from the group consisting of a lubricant, filler, binder, disintegrant, glidant, and solubilizer. In some embodiments, the matrixing agent comprises a hydrophilic polymer.

The controlled-release dosage form may be formed from the mixture, for example, by direct compression, or by wet or dry granulation followed by compression.

### DEFINITIONS

As used herein, the singular forms “a”, “and”, and “the” include plural referents unless the context clearly dictates otherwise. Thus, for example, reference to “anabaseine compound” includes a plurality of such compounds, reference to an “nAChR agonist” or “anabaseine agonist” includes a plurality of such agonists, reference to an “nAChR antagonist” or “anabaseine antagonist” includes a plurality of such antagonists and reference to “the nicotine acetylcholine receptor” includes reference to one or more receptors and equivalents thereof known to those skilled in the art, and so forth.

As utilized in accordance with the present disclosure, the following tent's, unless otherwise indicated, shall be understood to have the following meanings:

“Active ingredient” refers to an anabaseine compound (e.g., an anabaseine agonist or an anabaseine antagonist).

“Anabaseine agonist” refers to an anabaseine compound that binds substantially specifically to a nicotinic cholinergic receptor (nAChR) and causes the receptor to be activated to provide a pharmacological effect. Typically, activation of a nAChR causes its associated ion channel to open, resulting in calcium influx and membrane depolarization. This definition includes anabaseine partial agonists that, when bound to a nAChR, are less likely than a pure nicotinic agonist such as acetylcholine to cause activation, but activation does occur at least part of the time.

“Anabaseine antagonist” refers to an anabaseine compound that binds substantially specifically to a nicotinic cholinergic receptor (nAChR) but fails to cause its associated ion channel to open. However, this failure of the channel to open in turn results in a pharmacological effect. This definition includes partial anabaseine antagonists, which are anabaseine compounds that, when bound to a nAChR, are less likely than a pure anabaseine antagonist to block activation, but blocked activation does occur at least part of the time.

The terms “anabaseine compounds” and “compounds” are used interchangeably herein to refer to compounds having anabaseine as a component of their chemical structure, and which bind to nAChR, resulting in a pharmacological effect. Examples of anabaseine compounds include, but are not limited to, those disclosed in U.S. Patent Publication No. 2005/0288333 (Kem, “Controlling Angiogenesis with Anabaseine Analogs”), U.S. Pat. No. 5,977,144 (Meyer et al., “Methods of Use and Compositions for Benzylidene and Cinnamylidene-Anabaseines”), and U.S. Pat. No. 5,741,802 (Kem et al., “Anabaseine Derivatives Useful in the Treatment of Degenerative Diseases of the Nervous System”), and any compounds encompassed by generic formulae disclosed therein (the contents of which are incorporated herein by reference in their entirety). In preferred embodiments, the anabaseine compounds are arylidene-anabaseine compounds, such as 3-arylidene-anabaseine. The compounds may be identified either by their chemical structure and/or chemical name. When the chemical structure and chemical name conflict, the chemical structure is determinative of the identity of the compound. The compounds may contain one or more chiral centers and/or double bonds and therefore, may exist as stereoisomers, such as double-bond isomers (i.e., geometric isomers), enantiomers or diastereomers. Accordingly, when stereochemistry at chiral centers is not specified, the chemical structures depicted herein encompass all possible configurations at those chiral centers including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including cyclic imine form, cyclic iminium form, amino-keto form, ammonium-ketone form, and mixtures thereof. Accordingly, the chemical structures depicted herein encompass all possible tautomeric forms of the illustrated compounds. The compounds also include isotopically labeled compounds where one or more atoms have an atomic mass different from the atomic mass conventionally found in nature. Examples of isotopes that may be incorporated into the compounds include, but are not limited to, ²H, ³H, ¹³C, ¹⁴C, ¹⁵N, ¹⁷O and ¹⁸O. Compounds may exist in unsolvated forms as well as solvated forms, including hydrated forms and as N-oxides. In general, the hydrated, solvated and N-oxide forms are within the scope of the present disclosure. Certain compounds may exist in multiple crystalline or amorphous forms. In general, all physical forms are equivalent for the uses contemplated herein and are intended to be within the scope of the present disclosure. Further, it should be understood, when partial structures of the compounds are illustrated, that brackets indicate the point of attachment of the partial structure to the rest of the molecule.

“Halo” means fluoro, chloro, bromo, or iodo.

“Pharmaceutically acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, butyric acid, propionic acid, hexanoic acid, cyclopentanepropionic acid, glycolic acid, pyruvic acid, lactic acid, valeric acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid, muconic acid, and the like, made by conventional chemical means; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like, made by conventional chemical methods. A non-limiting example of a pharmaceutically acceptable salt of DMXBA is the dihydrochloride salt, i.e., (E)-3-(2,4-dimethoxybenzylidene)-3,4,5,6-tetrahydro-2,3′-bipyridine dihydrochloride.

“Pharmaceutically acceptable carrier” refers to a diluent, adjuvant, excipient or vehicle with which a compound is administered.

“Pharmaceutical composition” as used herein refers to at least one anabaseine compound (e.g., anabaseine agonist or anabaseine antagonist) and a pharmaceutically acceptable carrier with which the at least one anabaseine compound is administered to a subject.

“Protecting group” refers to a grouping of atoms that when attached to a reactive functional group in a molecule masks, reduces or prevents reactivity of the functional group. Examples of protecting groups can be found in Greene et al., “Protective Groups in Organic Chemistry”, (Wiley, 4th ed. 2007) and “Compendium of Synthetic Organic Methods”, Vols. 1-12 (John Wiley and Sons, 1971-2009). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

The term “alkyl” refers to the radical of saturated aliphatic groups, including straight-chain alkyl groups, and branched-chain alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C1-C30 for straight chain, C3-C30 for branched chain), preferably 26 or fewer, and more preferably 20 or fewer, and still more preferably 4 or fewer.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate.

The term “alkyl” also includes unsaturated aliphatic groups analogous in length and possible substitution to the alkyls described above, but that contain at least one double or triple bond respectively. An “alkylaryl” moiety is an alkyl substituted with an aryl (e.g., phenylmethyl (benzyl)).

The terms “alkoxy,” “aminoalkyl” and “thioalkoxy” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates propargyl group.

The term “aralkyl” means an aryl group that is attached to another group by a (C1-C6) alkylene group. Aralkyl groups may be optionally substituted, either on the aryl portion of the aralkyl group or on the alkylene portion of the aralkyl group, with one or more substituents.

The term “aryl” as used herein, refers to the radical of aryl groups, including 5- and 6-membered single-ring aromatic groups that may include from zero to four heteroatoms (heteroaryl), for example, benzene, pyrrole, furan, thiophene, imidazole, triazole, tetrazole, pyrazole, pyridine, pyrazine, pyridazine and pyrimidine, and the like. Aryl groups also include polycyclic fused aromatic groups such as naphthyl, quinolyl, indolyl, benzoxazolyl, benzothiazolyl and the like.

Those aryl groups having heteroatoms in the ring structure may also be referred to as “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryl oxycarbonyloxy, carboxylate, alkyl carbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, halogenated alkyl (including trifluoromethyl, difluoromethyl and fluororomethyl), halogenated alkoxy (including trifluoromethoxy, difluoromethoxy and fluororomethoxy), cyano, azido, heterocyclyl, alkylaryl, arylalkyl or an aromatic or heteroaromatic moiety. Aryl groups can also be fused or bridged with alicyclic or heterocyclic rings which are not aromatic so as to form a polycycle (e.g., tetralin).

The term “cyclyl” refers to a hydrocarbon 3-8 membered monocyclic or 7-14 membered bicyclic ring system having at least one non-aromatic ring, wherein the non-aromatic ring has some degree of unsaturation. Cyclyl groups may be optionally substituted with one or more substituents. In one embodiment, 0, 1, 2, 3, or 4 atoms of each ring of a cyclyl group may be substituted by a substituent. The term “cycloalkyl” refers to a hydrocarbon 3-8 membered monocyclic or 7-14 membered bicyclic ring system having at least one saturated ring. Cycloalkyl groups may be optionally substituted with one or more substituents. In one embodiment, 0, 1, 2, 3, or 4 atoms of each ring of a cycloalkyl group may be substituted by a substituent. Cycloalkyls can be further substituted, e.g., with the substituents described above. Preferred cyclyls and cycloalkyls have from 3-10 carbon atoms in their ring structure, and more preferably have 3, 4, 5, 6 or 7 carbons in the ring structure. Those cyclic groups having heteroatoms in the ring structure may also be referred to as “heterocyclyl,” “heterocycloalkyl” or “heteroaralkyl.” The aromatic ring can be substituted at one or more ring positions with such substituents as described above.

The terms “polycyclyl” or “polycyclic radical” refer to the radical of two or more cyclic rings (e.g., cycloalkyls, cycloalkenyls, cycloalkynyls, aryls, heteroaryls, and/or heterocyclyls). In some cases, two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, halogenated alkyl (including trifluoromethyl, difluoromethyl and fluororomethyl), halogenated alkoxy (including trifluoromethoxy, difluoromethoxy and fluororomethoxy), cyano, azido, heterocyclyl, alkyl, alkylaryl, or an aromatic or heteroaromatic moiety.

The term “haloalkyl” is intended to include alkyl groups as defined above that are mono-, di- or polysubstituted by halogen, e.g., fluoromethyl and trifluoromethyl.

The term “halogen” designates —F, —Cl, —Br, or —I.

The term “hydroxyl” means —OH.

The term “heteroatom” as used herein means an atom of any element other than carbon or hydrogen. Preferred heteroatoms are nitrogen, oxygen, sulfur and phosphorus.

The term “methyl” refers to a CH₃ group. In a preferred embodiment, R² at position 4, R³ at position 5 and R⁴ at position 6 on the tetrahydropyridyl ring are methyl groups either singly substituted, for example, R² is a methyl group or each is substituted with methyl and are in (S)- or (R)-alpha or beta) enantiomeric form.

The term “mercapto” refers to a SH group.

The term “sulfhydryl” or “thiol” means —SH.

The compounds of the invention encompass various isomeric forms. Such isomers include, e.g., stereoisomers, e.g., chiral compounds, e.g., diastereomers and enantiomers.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

Furthermore the indication of configuration across a carbon-carbon double bond can be “Z” referring to what is often referred to as a “cis” (same side) conformation whereas “E” refers to what is often referred to as a “trans” (opposite side) conformation. Regardless, both configurations, cis/trans and/or Z/E are contemplated for the compounds for use in the present invention.

With respect to the nomenclature of a chiral center, the terms “S” and “R” configuration are as defined by the IUPAC Recommendations. As to the use of the terms, diastereomer, racemate, epimer and enantiomer, these will be used in their normal context to describe the stereochemistry of preparations.

Natural amino acids represented by the compounds utilized in the present invention are in the “L” configuration, unless otherwise designated. Unnatural or synthetic amino acids represented by the compounds utilized in the present invention may be in either the “D” or “L” configurations. Similarly glycosidic bonds may be in either alpha- or beta-configuration.

Another aspect is a radiolabeled compound or a compound containing one or more stable isotope atoms of any of the formulae delineated herein. Such compounds have one or more radioactive atoms (e.g., ³H, ¹⁴C, ³⁵S, ³²P) or one or more stable isotope atoms (e.g., ²H, ¹³C) introduced into the compound. Such compounds are useful for drug metabolism studies and diagnostics, as well as therapeutic applications.

The term “prodrug” includes compounds with moieties, which can be metabolized in vivo. The anabaseine compounds used in the formulations and methods of the invention may be prodrugs. Generally, the prodrugs are metabolized in vivo by esterases or by other mechanisms to active drugs. Examples of prodrugs and their uses are well known in the art (See, e.g., Berge et al., 1977, “Pharmaceutical Salts”, J. Pharm. Sci. 66:1-19; Silverman, 2004, “The Organic Chemistry of Drug Design and Drug Action”, Second Ed., Elsevier Press, Chapter 8, pp. 497-549). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halogen, or methoxy substituents) aryl and aryl-lower alkyl esters, amides, lower-alkyl amides, di-lower alkyl amides, and hydroxy amides. Preferred prodrug moieties are propionoic and succinic acid esters, acyl esters and substituted carbamates. Prodrugs which are converted to active forms through other mechanisms in vivo are also included.

“Substituted” refers to a group in which one or more hydrogen atoms are independently replaced with the same or different substituent(s). Typical substituents include, but are not limited to, alkyl and N,N-dialkylamino.

“Treat”, “treating” and “treatment” all refer to obtaining a desired pharmacologic and/or physiologic effect, such as preventing, delaying onset of, eliminating, or reducing the severity of one or more symptoms associated with a disease or condition caused or exacerbated by a defect in, and/or malfunctioning of, a nicotinic receptor (such as alpha7 nAChR). The defective and/or malfunctioning nicotinic receptor(s) may be a neuronal nicotinic receptor or a non-neuronal nicotinic receptor, or both types of receptors may be defective and/or malfunctioning, causing or exacerbating the disease or condition. For example, the physiological effect can be an improvement in a cognitive deficit (such as a deficit in learning and/or memory), for example, through increased brain cholinergic transmission; or stimulation of angiogenesis and/or vasculogenesis or the inhibition of angiogenesis. The effect may be prophylactic in terms of completely or partially preventing a disease or symptom thereof and/or may be therapeutic in terms of a partial or complete cure for a disease and/or adverse effect attributable to the disease. For embodiments of the invention involving improving cognitive dysfunction, “treatment” as used herein includes any treatment of a disease in a mammal, such as a human, and includes: (a) preventing or delaying onset of a disease or condition (e.g., preventing or delaying the onset of learning and/or memory impairments) from occurring in a subject who may be predisposed to the disease but has not yet been diagnosed as having it; (b) inhibiting the disease, e.g., arresting its development; or (c) relieving the disease (e.g., improving learning and/or cognitive deficits). For embodiments of the invention involving stimulation of angiogenesis, “treatment” as used herein includes any treatment of a disease in a mammal, such as a human, and includes: (a) preventing or delaying onset of a disease or condition (e.g., preventing the loss of a skin graft or a re-attached limb due to inadequate vascularization) from occurring in a subject who may be predisposed to the disease but has not yet been diagnosed as having it; (b) inhibiting the disease, e.g., arresting its development; or (c) relieving the disease (e.g., enhancing the development of a “bio-bypass” around an obstructed vessel to improve blood flow to an organ or enhancing wound healing in an ischemic limb). Stimulation of angiogenesis and/or vasculogenesis can be employed for a subject having a disease or condition amenable to treatment by increasing vascularity and increasing blood flow. For embodiments of the invention involving inhibition of angiogenesis, “treatment” as used herein includes any treatment of a disease in a mammal, such as a human, and includes: (a) preventing or delaying onset of a disease or condition (e.g., preventing proliferative retinopathy) from occurring in a subject who may be predisposed to the disease but has not yet been diagnosed as having it; (b) inhibiting the disease (e.g., inhibiting further growth of a tumor); or relieving the disease. Reduction of angiogenesis and/or vasculogenesis can be employed for a subject having a disease or condition amenable to treatment by reducing angiogenesis.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms, “subject”, “patient” or “individual” are used interchangeably herein, and refer to a mammalian subject to be treated, with human patients being preferred. In some cases, the methods of the invention find use in experimental animal models, in veterinary application, and in the development of animal models for disease, including, but not limited to, rodents including mice, rats, and hamsters; and primates.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Therapeutically effective amount” means the amount of a compound that, when administered to a subject to treat a disease or condition caused or exacerbated by a defect in, and/or malfunctioning of, a neuronal nicotinic acetylcholine receptor (such as alpha7 nAChR) or non-neuronal nicotinic acetylcholine receptor, is sufficient to effect such treatment. The “therapeutically effective amount” will vary depending on the compound, the severity of the disease or condition associated with a defect in, and/or malfunctioning of, a nicotinic receptor, and the age, weight, etc., of the patient to be treated.

In addition to neurons, alpha7 nAChRs have also been found on non-neuronal cells within the nervous system (for example, astrocytes and microglia) and outside the nervous system; e.g., macrophages, bronchial epithelium and vascular endothelium. Alpha7 receptors on peripheral macrophages, when stimulated by appropriate agonists, inhibit the secretion of cytokines, including tumor necrosis factor alpha (TNF), which cause inflammation. Similarly, stimulation of alpha7 nAChRs in vascular endothelium enhances the formation of new blood vessels (angiogenesis), an important process in wound healing. On the other hand, proliferation of certain small cell lung cancers expressing primarily alpha7 nAChRs can be stimulated by nicotinic agonists and possibly inhibited with certain nicotinic antagonists. Thus, besides being implicated as useful therapeutic targets for treating nervous system disorders such as Alzheimer's disease and schizophrenia, alpha7 nAChRs on non-neuronal cells may also be therapeutic targets for treating other disease states involving inflammation, trauma, deficient or excessive angiogenesis, and abnormal proliferation such as cancer (Cai, B. et al., Oct. 17, 2008, J. Cell Mol. Med., Epub; Kox, M. et al., 2009, Biochem. Pharmacol., 78(7):863-872; Rosas-Ballina, M. et al., 2009, Mol. Med., 15(7-8):195-202; van Westerloo, D. J. et al., 2006, Gastroenterology, 130(6):1822-1830; van Maanen, M. A. et al., 2009, Arthritis Rheum., 60(5):1272-1281; De Rosa, M. J. et al., Jul. 24, 2009, Life Sci., Epub; Paleari, L. et al., Jul. 16, 2009, Drug Discov. Today, Epub).

The term “anti-angiogenic activity”, as used herein, refers to the inhibition and/or moderation of angiogenesis.

The term “angiogenesis-associated disease”, as used herein, refers to certain pathological processes in mammals such as humans where angiogenesis is abnormally prolonged. For example, angiogenesis-associated diseases include diabetic retinopathy, chronic inflammatory diseases, rheumatoid arthritis, dermatitis, psoriasis, stomach ulcers, tumors and the like.

The term “controlled-release” refers to a formulation, dosage form, or region thereof from which release of a beneficial agent is not immediate, i.e., with a “controlled-release” dosage form, administration does not result in immediate release of the beneficial agent in an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled-release” as used herein includes timed release, sustained-release, and delayed release formulations.

The term “sustained-release” (synonymous with “extended-release”) is used in its conventional sense to refer to a formulation, dosage form, or region thereof that provides for gradual release of a beneficial agent over an extended period of time, and that preferably, although not necessarily, results in substantially constant blood levels of the agent over an extended time period.

In the present context, the terms “controlled-release” and “modified-release” are intended to be equivalent terms covering any type of drug release from a composition prepared according to the invention, which drug release is appropriate to obtain a specific therapeutic or prophylactic response after administration to a subject. A person skilled in the art knows how controlled-release/modified-release differs from the release of plain (immediate release) tablets or capsules. The terms “release in a controlled manner” or “release in a modified manner” have the same meaning as stated above. The terms include slow release (that results in a lower Cₘₐₓ, and later tₘₐₓ, but t_{1/2} is unchanged), extended-release (that results in a lower Cₘₐₓ, later tₘₐₓ, but apparent t_{1/2} is longer); delayed release (that result in an unchanged Cₘₐₓ, but lag time and, accordingly, tₘₐₓ is delayed, and t_{1/2} is unchanged) as well as pulsatile release, burst release, sustained-release, prolonged release, chrono-optimized release, fast release (to obtain an enhanced onset of action) etc. Included in the terms is also e.g., utilization of specific conditions within the body e.g., different enzymes or pH changes in order to control the release of the drug substance.

The term “naturally occurring” refers to a compound or composition that occurs in nature, regardless of whether the compound or composition has been isolated from a natural source or chemically synthesized.

As used herein, “binding” refers to the formation of a complex involving a receptor (e.g., an nAChR such as alpha 7) and a ligand, and “binding affinity” refers to a compound's capacity to bind to a receptor. Binding affinity may be quantified, for example, by Kᵢ.

A compound may exhibit “selective” binding, by which is meant that the compound's affinity for binding to one or more particular receptor(s) is greater than the compound's affinity for binding to one other receptor, multiple other receptors, or all other receptors. For a compound that exhibits selective binding, therefore, the binding constant Kᵢ for the compound binding with one receptor is lower than the Kᵢ for the compound binding with one or more other receptor(s). For example, a compound that is selective for receptor “A” over receptor “B” will have a binding constant ratio Kᵢ(A)/Kᵢ(B) that is less than 1/1.

As used herein, the term “drug” means a compound intended for use in diagnosis, cure, mitigation, treatment, or prevention of disease in man or other animals. For example, the drug may be one or more anabaseine compounds, such as DMXBA.

In this context, the term “dosage form” means the form in which the drug is delivered to the subject. This can be parenteral, topical, capsule, tablet, oral (e.g., liquid or dissolved powder), suppository, inhalation, transdermal, etc.

In this context, the term “erosion” or “eroding” means a gradual breakdown of the surface of a material or structure, for example of a tablet or capsule or the coating of a tablet or capsule.

In the present context, the terms “modified-release” or “controlled-release” indicate that efforts have been made to deliberately or actively target a specified release of an active drug substance from a drug composition. Essentially, any type of altered release pattern is included in these terms. The terms could theoretically be applied to any therapeutic administration of a drug, but in preferred embodiments it is limited to oral administration of solid dosage forms. The terms modified- or controlled-release cover a number of sub-terms such as, e.g., slow-release, extended-release, prolonged-release, sustained-release, delayed-release, pulsed-release and also site-specific releases like: buccal-release, gastrointestinal-release, stomach-release, intestinal-release, duodenal-release, jejunum-release, ileum-release and colon-release.

For site-specific releases, including delayed release and pulsed release, a fast release at a specific site or at predetermined time would normally be attractive. The delaying factor can be a coating or a sensitive matrix withholding release until a certain time had passed, pH had changed, the composition had been subject to enzymes, or some other site-specific external factor is present. Ion-exchange systems would also be considered members of this class.

In the group of slow-release, the controlling factors most often are either a semi-permeable coating or a gelling barrier of some kind (e.g., a gel-forming polymer). The systems can be mono-particulate (matrix) or multi-particulate (granules, pellets, beads, etc.), for example.

Reference will now be made in detail to certain preferred methods of treatment, compounds, methods for production, and methods of administering these compounds. The invention is not limited to those preferred compounds and methods but rather is defined by the claim(s) issuing herefrom.

### Anabaseine Compounds

The present invention provides controlled-release formulations (dosage forms) of anabaseine compounds, such as 3-(2,4-dimethoxybenzylidene)-anabaseine (also known as DMXBA, DMXB, and GTS-21), and methods for the treatment or prevention of various diseases or conditions caused or exacerbated by defects in and/or malfunctioning of nicotinic acetylcholine receptors. In these methods, the disease or condition is treated or prevented by administering an effective amount of one or more controlled-release formulations of anabaseine compounds to a subject, upon which the anabaseine compound or compounds produce specific plasma levels and/or brain levels of the compound(s) or their active metabolite(s) in the subject. Also provided are methods for providing desired plasma levels and/or brain levels of anabaseine compounds or metabolites thereof in a subject. The anabaseine compound may be an anabaseine agonist or an anabaseine antagonist.

The defective and/or malfunctioning nicotinic acetylcholine receptor type(s) may be, for example, a neuronal nicotinic receptor or a non-neuronal nicotinic receptor, or both types may be defective and/or malfunctioning. In some embodiments, the nicotinic acetylcholine receptors are neuronal nicotinic acetylcholine receptors (e.g., brain nicotinic receptors such as alpha7-containing receptors, alpha4(2)beta2(3), or alpha4(3)beta2(2)), and the disease or condition is Alzheimer's disease, schizophrenia, Parkinson's disease, attention deficit-hyperactivity disorder, or another disease or condition caused or exacerbated by a defect in, and/or malfunctioning of, a neuronal nicotinic acetylcholine receptors. In some embodiments, the nicotinic acetylcholine receptors are non-neuronal nicotinic acetylcholine receptors, and the disease or condition is an inflammatory disorder, trauma, deficient angiogenesis, excessive angiogenesis, abnormal cell proliferation (e.g., cancer), or another disease or condition caused or exacerbated by a defect in, and/or malfunctioning of, a non-neuronal nicotinic acetylcholine receptor.

Although nicotinic cholinergic receptors (nAChRs) in the brain have long been recognized as being important in mediating the euphoric effects of nicotine, they attracted additional interest when significant nAChR deficits, later identified as primarily of the alpha4beta2 receptor subtype, were discovered in postmortem brain samples from patients with Alzheimer's disease (Nordberg A. and B. Winblad, 1986, Neurosci. Lett., 72:115-121; Whitehouse, P. J. et al., 1986. Brain Res., 371:146-151). Mammalian nAChRs are pentam eric ligand-gated ion channels that, upon activation, allow the movement of cations including calcium across the cell membrane. Besides causing membrane depolarization, nAChR (especially the alpha7 type)-mediated influx of calcium into the cell stimulates several signal transduction pathways (T. Kihara et al., 2001, J. Biol Chem., 276:13541-13546; F. A. Dajas-Bailador et al., 2002, J. Neurochem., 80:520-530). A variety of the nAChR subtypes are now known to be present in the mammalian brain. The two most abundant brain nAChRs are the alpha4beta2 and homomeric alpha7 subtypes. The former contributes >90% of the high-affinity binding sites for nicotine in the rat brain (G. M. Flores et al., 1992, Mol. Pharmacol., 41:31-37). The low-nicotine-affinity alpha7 nAChR is recognized by its nanomolar affinity for alpha-bungarotoxin (BTX) (M. J. Marks and A. C. Collins, 1982, Mol. Pharmacol., 22:554-564).

Anabaseine is an animal toxin that, like nicotine, stimulates all nAChRs (W. R. Kem et al., 1997, J. Pharmacol. Exp. Ther., 283:979-992). DMXBA, dimethoxybenzylidene)-anabaseine dihydrochloride, also known as GTS-21, a synthetic benzylidene derivative of anabaseine, selectively stimulates alpha7 subunit-containing nAChRs (C. M. de Fiebre et al., 1995, Mol. Pharmacol., 47:164-171; E. M. Meyer et al., 1998, J. Pharmacol. Exp. Ther., 287:918-925). Its efficacy (maximum effect) for activating the rat alpha7 receptor is 59% of that observed for acetylcholine and anabaseine, which are full agonists (Kem et al., 2004). DMXBA efficacy at the human alpha7 receptor is only 23% of the acetylcholine maximal response (Meyer et al., 1998; Kem et al., 2004). DMXBA displays neuroprotective properties (E. J. Martin et al., 1994, Drug Dev. Res., 31:134-141; T. Kihara et al. 1997, Ann. Neurol., 42:159-163; S. Shimohama et al., 1998, Brain Res., 779:359-363).

Anabaseine's non-aromatic, tetrahydropyridyl ring imine double bond is conjugated with pi-electrons of the 3-pyridyl ring. The imine nitrogen is a weaker base than the pyrrolidinyl nitrogen of nicotine (Yamamoto, et al., 1962, Agr. Biol Chem., 26:709). Considerable evidence (Barlow and Hamilton, 1962, Brit. J. Pharmacol., 18:543) exists that the non-aromatic ring nitrogen of nicotine must be protonated (cationic) in order to avidly bind to the skeletal muscle nicotinic receptor and activate the opening of its channel. At physiological pH, anabaseine also exists in a hydrolyzed ammonium-ketone form as well as the cyclic imine (unionized) and cyclic iminium (monocationic) forms. Kem (“Nemertine Toxins,” in Animal Toxins, Tools in Cell Biology, eds., H. Rochat and M.-F. Martin-Eauclaire, Chapman and Hail, pp. 57-73) has determined that anabaseine acts as a central nicotinic receptor agonist primarily through its cyclic iminium form.

Suitable anabaseine compounds include, but are not limited to, anabaseine agonists and anabaseine antagonists of formula I and/or formula II as described in U.S. Patent Publication No. 2005/0288333 (Kem, “Controlling Angiogenesis with Anabaseine Analogs”) and shown in FIGS. 4A-4C and FIG. 5 herein. Accordingly, in some embodiments, the anabaseine compounds have the chemical structure of formula I of U.S. Patent Publication No. 2005/0288333, shown in FIGS. 4A-4C herein, or a pharmaceutically acceptable salt thereof; wherein R¹ is hydrogen acetoxy, acetamido, amino, dimethylcarbamate, dimethylaminopropoxy, hydroxyl, methoxy, methyl, propyl, ethyl, isopropoxy, trifluoromethoxy or thiomethoxy or C₁-C₄ alkyl; and R₂ is hydrogen, methyl, propyl, ethyl, hydrogen, (S,R)-methyl, S- or R-methyl, (S,R)-propyl, S- or R-propyl, (S,R)-ethyl, S- or R-ethyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl or the substituent of FIG. 4B herein, wherein R³, R⁴, and R⁵ are each selected from hydrogen, methyl, propyl, ethyl, methoxy, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro; or R² is ═CHCH═CHZ, wherein Z is the substituent shown in FIG. 4C herein, wherein R⁶, R⁷, and R⁸ are selected from the group consisting of hydrogen, methyl, propyl, ethyl, methoxy, cyano-, phenoxy, phenyl, pyridyl or benzyl, C₁-C₄ alkyl optionally substituted with N,N-dialkylamino having 1 to 4 carbons in each of the alkyls, C₁-C₆ alkoxy optionally substituted with N,N-dialkylamino having 1 to 4 carbons in each of the alkyls, carboalkoxy having 1 to 4 carbons in the alkoxy, amino, acylamino having 1 to 4 carbons in the acyl, cyano, N,N-dialkylamino having 1 to 4 carbons in each of the alkyls, halo, hydroxyl, and nitro; or a pharmaceutically acceptable salt, solvate, clathrate, stereoisomer, enantiomer, prodrug or combinations thereof, wherein the compound of Formula I and/or II functions as either an anabaseine agonist or an anabaseine antagonist. In the embodiments of the invention involving methods for induction of angiogenesis in a mammal, the compounds of Formula I and/or II that function as anabaseine agonists can be administered. In the embodiments of the invention involving methods of reducing angiogenesis in a mammal, the compounds of Formula I and/or II that function as anabaseine antagonists can be administered.

In a preferred embodiment, R² at position 4, R³ at position 5 and R⁴ at position 6 on the tetrahydropyridyl ring are methyl, propyl, ethyl groups either singly substituted, for example, R² is a methyl group or each is substituted with methyl and are in (S)- or (R)-(alpha or beta) enantiomeric form.

Examples of anabaseine compounds that may be used in the compositions and methods of the invention include, but are not limited to:
3-(4-Methoxybenzylidene)-anabasein
3-(4-Nitrobenzylidene)-anabaseine;
3-(4-Cyanobenzylidene)-anabaseine;
3-(4-Hydroxybenzylidene)-anabaseine;
3-(4-Chlorobenzylidene)-anabaseine;
3-(4-Aminobenzylidene)-anabaseine;
3-(4-Dimethylaminopropoxy-benzylidene)-anabaseine;
3-(2-Methoxybenzylidene)-anabaseine;
3-(3-Methoxybenzylidene)-anabaseine;
DMXBA, 3-(2,4-Dimethoxybenzylidene)-anabaseine;
3-(3-Methoxy-4-hydroxybenzylidene)-anabaseine;
6′-Methyl anabaseine;
2′-Methyl anabaseine;
4′-Methyl anabaseine;
3-(2,4,6-Trimethoxybenzylidene)-anabaseine;
3-(2,4-Dichlorobenzylidene)-anabaseine;
3-(2,4-Dim ethylbenzylidene)-anabaseine;
3-(2,4,6-Trimethylbenzylidene)-anabaseine;
3-(2-Furylmethylidene)-anabaseine;
3-(2-Furylpropenylidene)-anabaseine;
3-(3-Furylmethylidene)-anabaseine;
3-(4-Methylbenzylidene)-anabaseine;
3-(2-Hydroxy-4-methoxybenzylidene)-anabaseine;
3-(2,4-Dihydroxybenzylidene)-anabaseine
3-(2,4-Dipropoxybenzylidene)-anabaseine;
3-(2,4-Diisopropoxybenzylidene)-anabaseine;
3-(2,4-Diisopentoxybenzylidene)-anabaseine;
3-(2-Hydroxy-4-isopentoxybenzylidene)-anabaseine;
6′-Methyl-3-(2,4-dimethoxybenzylidene)-anabaseine;
1-Methyl-3-(2,4-dimethoxybenzylidene)-anabaseine trifluoroacetate;
5′-Methylanabaseine;
3-(2-Methoxy-4-hydroxybenzylidene)-anabaseine;
2-Phenyl-3-(2,4-dimethoxybenzylidene)-4,5,6-trihydropyridine;
and DMACA, 3-(4-Dimethylaminocinnamylidene)-anabaseine;
   active metabolites of any of the foregoing, and pharmaceutically acceptable salts of the aforementioned compounds or metabolites.

Accordingly, in some embodiments, the anabaseine compounds have the chemical structure of formula II of U.S. Patent Publication No. 2005/0288333, which is shown in FIG. 5 herein, or a pharmaceutically acceptable salt thereof; wherein R¹ is hydrogen, methyl, propyl, ethyl, acetoxy, acetamido, amino, dimethylcarbamate, dimethylaminopropoxy, hydroxyl, methoxy, isopropoxy, trifluoromethoxy or thiomethoxy or C₁-C₄ alkyl; and R² is hydrogen, methyl, propyl, ethyl, (S,R)-methyl, S- or R-methyl, (S,R)-propyl, S- or R-propyl, (S,R)-ethyl, (S)- or (R)-ethyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl or, wherein R³, R⁴, and R⁵ are each selected from hydrogen, methyl, propyl, ethyl, methoxy, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro; wherein the compound of Formula I functions as either an anabaseine agonist or an anabaseine antagonist.

In a preferred embodiment, R² at position 4, R³ at position 5 and R⁴ at position 6 on the tetrahydropyridyl ring are methyl, propyl, ethyl groups either singly substituted, for example, R² is a methyl group or each site is substituted with methyl and are in (S)- or (R)-(alpha or beta) enantiomeric form.

Appropriate substituents on the tetrahydropyridyl ring portions of anabaseine compounds can be selected for desired alpha7 selectivity, either when done separately or in combinations. Exemplary substitutions are: R² at position 4, R³ at position 5, and R⁴ at position 6 on the tetrahydropyridyl ring are alkyl groups (e.g., methyl groups) either singly substituted, for example, R² is an alkyl group or each site is substituted with an alkyl group and are in (S)- or (R)-(alpha or beta) enantiomeric form.

Certain substituents also determine alpha7 receptor efficacy: Some substituents increase efficacy over benzylidene-anabaseines such as DMXBA while other reduce efficacy to essentially zero, thereby creating a new group of alpha7 nAChR antagonists.

Potential applications of these alpha7 agonists and antagonists based on the anabaseine structure include therapeutic treatments for neurodegenerative diseases and nicotinic receptor involved addictions as well as potential for development as antiproliferation drugs. In particular, it is shown that altering anabaseine compound polarity and ionization can permit drug application and localization to the peripheral (blood and interstitial fluid) compartments without significant entry into the central nervous system.

In addition to CNS applications, some anabaseine compounds can provide therapeutic agents that selectively stimulate peripheral alpha7 receptors expressed on non-neuronal cells such as macrophages, vascular endothelium and bronchial epithelium, which are peripheral cells known to express functional alpha7 nAChRs. When macrophage alpha7 receptors are stimulated, their secretion of inflammatory cytokines such as TNF is inhibited. These cytokines are known to exacerbate an inflammatory response when overproduced and not efficiently removed from the system. It is believed that alpha7 nicotinic receptor agonists may be useful in stimulating neoangiogenesis in wound healing and other conditions in which there is inadequate tissue perfusion. New tissue requires a robust blood supply in order to function efficiently and tissue lacking sufficient oxygenation may become necrotic. Development of new blood vessels is of prime importance in recovery of damaged heart tissue. The brain is the site of several types of insults, including stroke, vascular dementia and there is a decrease in number of microvessels in the aging brain. In selected cases therefore, it may be beneficial to target cerebral microvessels in the basal lamina with the agents of the present invention in order to stimulate neoangiogenesis and increase blood flow and distribution in the brain.

Inhibition of angiogenesis would be desirable in certain medical conditions, such as in tumor cell proliferation and in some forms of retinal (macular) degeneration. Alpha7 nAChR antagonists could be useful in inhibiting angiogenesis, as new blood vessel growth is necessary for growth of solid tumors. An anabaseine alpha7 nAChR antagonist that is polar, and/or ionized and/or conjugated to another inactive molecule such as a complex carbohydrate or a polyethylene glycol that confers on the molecule pharmacokinetic advantages and limits its diffusion to the compartment of administration may be useful as angiogenesis inhibitor in treating certain conditions. Such an anabaseine type alpha7 nAChR antagonist could also be directly administered into the arterial blood perfusing the tumor to achieve even greater selectivity of action.

Anabaseine compounds can be useful in a number of applications, particularly in treatment of diseases where it is advantageous to upregulate alpha7 nicotinic receptor activity. Loss of neuronal alpha7 receptors occurs in the progression of Alzheimer's disease and there is deficient expression of this receptor subtype in schizophrenia. It has been shown that chronic administration of alpha7 agonists such as DMXBA can lead to an increased expression of functional alpha7 receptors on cell surfaces. Thus, chronic administration of an alpha7-selective drug may have an even greater effect than before up-regulation in alpha7 number and responsiveness has occurred. In contrast to alpha7 selective ligands, alpha4beta2 receptor ligands generally cause a down-regulation of overall responsiveness of a cell while at the same time there may be an increase in alpha4beta2 receptor number. Thus, chronic administration of alpha4beta2 agonists is more likely to cause tolerance. An up-regulation in responsiveness may be exhibited by the anabaseine compounds used in the controlled-release formulations of the invention, either alone or in combination, in appropriate pharmaceutically acceptable forms.

The anabaseine compounds used in the formulations and methods of the invention may be selective ligands (agonists or antagonists) of alpha7 nicotinic receptors, which have little or no activity with respect to other nACh receptor subtypes, particularly alpha4beta2 receptors. Exemplary anabaseine compounds include compounds with substituents on one or more of the three ring systems present, i.e., pyridyl, tetrahydropyridyl and 3-arylidene. It has been discovered that selection of a particular substituent to be placed in one of these rings can improve selectivity of binding for the alpha7 receptor and can also determine whether the occupied receptor will be activated or inhibited. For example, substitutions expected to provide these properties include acetamido, acetoxy, alkoxy, alkyl, amino, aryl, benzofuran-2-ylmethylene, benzyl, carbamate, dimethylaminoalkoxy, modified glucuronidyl and 1H-indol-2-ylmethylene groups. Substitution at the alpha- or beta-oriented sites at positions 4, 5, and 6 of the tetrahydropyridyl ring form chiral products that display significantly improved alpha7 receptor selectivity in comparison with the corresponding racemic substituted compounds. Multiple substituents on one or more ring portions of these anabaseine compounds are expected to provide even greater selectivity than when substituents are made individually.

In a preferred embodiment, R² at position 4, R³ at position 5 and R⁴ at position 6 on the tetrahydropyridyl ring are alkyl groups (e.g., methyl groups) either singly substituted, for example, R² is an alkyl group or each is substituted with an alkyl group and single substitutions are in (S)- or (R)-(alpha or beta) enantiomeric form.

In another preferred embodiment, R² at position 4, R³ at position 5, and R⁴ at position 6 on the tetrahydropyridyl ring are substituted with one or more of: methyl, propyl, and ethyl groups.

The compounds of Formula I may be prepared by chemical synthesis according to the methods disclosed in U.S. Pat. No. 5,616,785, issued May 14, 1996; U.S. Pat. No. 5,741,802, issued Apr. 21, 1998; U.S. Pat. No. 5,977,144, issued Nov. 2, 1999; and U.S. Pat. No. 6,630,491, issued Oct. 7, 2003. Briefly, the compounds of Formula I and/or II wherein R² is other than ═CH—X or ═CHCH═CH—Z may be prepared by reacting the appropriate protected 2-piperidone with an appropriate pyridyl lithium or phenyl lithium. Pyridyl lithium may be prepared from the corresponding bromopyridine (H. Gilman, et al., 1951, J. Org. Chem., 16:1485). Typically, the pyridyl lithium, which is freshly prepared, is used in the condensation in an inert solvent, e.g., dry ether. The reaction is usually complete within a few hours. The reaction mixture is then acidified and the product is isolated by solvent extraction and purified by, for example, recrystallization.

The compounds of Formula I and/or II wherein R² is ═CH—X may be prepared from anabaseine. In general, a solution of anabaseine (or its dihydrochloride) in acidic ethanol is treated with about two molar equivalents of an aldehyde (X—CHO), and the resulting mixture is heated to approximately 70 degrees C. for about 16 hours. The compounds of Formula. I and/or II can be isolated and purified by standard techniques such as chromatography and recrystallization.

Although the above acidic reaction conditions are generally satisfactory, more basic reaction conditions or buffered (usually with acetic acid-sodium acetate) conditions may be required in the case of reacting aldehydes bearing an electron-withdrawing group such as nitro. Thus, a basic agent can also be used in the mixed aldol-type condensation.

The compounds of Formula I and/or II wherein X is substituted or unsubstituted phenyl can adopt two conformations about the double bond at the 3-position. Although the E isomer is preferred, a Z isomer may occur. Both E and Z isomers are considered to be within the purview of the present invention.

The compounds of Formula I and/or II wherein R² is ═CHCH═CH—Z may be prepared as disclosed in U.S. Pat. No. 5,911,144, issued Nov. 2, 1999, which is herein incorporated by reference.

The compounds of Formula I and/or II in their free base form will form acid addition salts, and these acid addition salts are non-toxic and pharmaceutically acceptable for therapeutic use. The acid addition salts can be prepared by standard methods, for example by combining a solution of anabaseine in a suitable solvent (e.g., water, ethyl acetate, acetone, methanol, ethanol or butanol) with a solution containing a stoichiometric equivalent of the appropriate acid (monocationic or dicationic salts may be prepared by adding one or two equivalents of acid per anabaseine compound free base). If the salt crystallizes or precipitates, it is recovered by filtration. Alternatively, it can be recovered by evaporation of the solvent or, in the case of aqueous solutions, by lyophilization. Of particular value are the sulfate, hydrochloride, hydrobromide, nitrate, phosphate, citrate, tartrate, pamoate, perchlorate, sulfosalicylate, benzenesulfonate, 4-toluenesulfonate and 2-naphthalenesulfonate salts. These acid addition salts are considered to be within the scope and purview of this invention.

The anabaseine compounds that may be used in the controlled-release formulations of the invention include arylidene-anabaseine compounds, such as 3-arylidene-anabaseine compounds. A large number of 3-arylidene-anabaseine compounds have been prepared (WO 2004/019943 and WO 2006/133303, which are incorporated herein by reference in their entirety) for potential use in treating neurodegenerative diseases, and particularly with the expectation that some compounds would bind to nicotinic alpha7 receptors. Many of these anabaseine compounds contain fused-ring heteroaromatic moieties attached through a methylene group to the 3-position of anabaseine without substitutions on the tetrahydropyridyl ring in the anabaseine molecule.

The one or more anabaseine compounds that may be used in the controlled-release formulations can be a cinnamylidene-anabaseine or benzylidene-anabaseine, for example. Examples of arylidene-anabaseines such as cinnamylidene-anabaseines and benzylidene-anabaseines include those disclosed in U.S. Pat. No. 5,977,144 (Meyer et al., “Methods of Use and Compositions for Benzylidene- and Cinnamylidene-Anabaseines”, which is incorporated herein by reference in its entirety), having a formula shown in FIGS. 6A-6B herein. Specific examples of such compounds are listed in Tables 1-3 of U.S. Pat. No. 5,977,144. Further examples of benzylidene-anabaseines that may be used in the controlled-release formulations are those disclosed in LeFrancois, S. E., 2004, “A Structure Activity Investigation of Benzylidene Anabaseine Interactions with the Alpha7 Nicotinic Acetylcholine Receptor,” A Dissertation, University of Florida, which is incorporated herein by reference in its entirety.

Other anabaseine compounds that may be used in the controlled-release formulations include the anabaseine-related compounds identified in U.S. Pat. No. 5,741,802 (Kem et al., “Anabaseine Derivatives Useful in the Treatment of Degenerative Diseases of the Central Nervous System”, which is incorporated herein by reference in its entirety).

### Controlled-Release (CR) Systems

As described herein, formulations of the anabaseine compound can be used to provide controlled-release (“controlled-release formulations”) in which the release of the one or more anabaseine compounds is controlled and regulated to allow less frequency of dosing or to improve the pharmacokinetic or toxicity profile of a given anabaseine compound or other active ingredient.

A controlled-release formulation as described herein may allow dosage once, twice, or three or more times daily in order to obtain a suitable therapeutic effect. Controlled-release may also include continuous and/or sustained-release, for example, as from an implantable device. Pulsatile release may also be desirable. Administration may comprise co-administration of more than one dosage unit, such as, e.g., 2-4 dosage units.

Controlled-release preparations are employed to control the duration of action of the one or more anabaseine compounds. Controlled-release preparations may be achieved by the use of controlled-release agents such as polymers to complex or adsorb the one or more anabaseine compounds. The controlled delivery may be exercised by selecting appropriate macromolecules (for example, polyesters, polyamino acids, polyvinyl pyrrolidone, ethylene vinyl acetate copolymers, methylcellulose, carboxymethylcellulose, and protamine sulfate) and the concentration of macromolecules as well as the methods of incorporation in order to control release. Another possible method to control the duration of action by controlled-release preparations is to incorporate the one or more anabaseine compounds into particles of a polymeric material such as polyesters, polyamino acids, hydrogels, poly (lactic acid) or ethylene vinylacetate copolymers. Alternatively, instead of incorporating the one or more anabaseine compounds into these polymeric particles, it is possible to entrap the one or more anabaseine compounds in microcapsules prepared, for example, by coacervation techniques or by interfacial polymerization, for example, hydroxymethylcellulose or gelatin-microcapsules and poly (methylmethacrylate) microcapsules, respectively, or in colloidal drug delivery systems, for example, liposomes, albumin microspheres, macroemulsions, nanoparticles, and nanocapsules or in macroemulsions. Such teachings are disclosed in Remington's Pharmaceutical Sciences (17th Ed., A. Oslo, ed., Mack, Easton, Pa., 1985).

In some embodiments, the one or more anabaseine compounds are prepared with carriers that will protect the one or more anabaseine compounds against rapid elimination from the body, such as a controlled-release matrix, implant, and/or microencapsulated delivery system. Biodegradable, biocompatible polymers can be used, such as ethylene vinylacetate copolymers, polyanhydrides, polyglycolic acid, collagen, polyorthoesters, and polylactic acid.

CR formulations of the drug may be processed by methods including but not limited to: direct compression (dry blend of drug with flowable excipients followed by compression), wet granulation (application of a binder solution to powder blend, followed by drying, sizing, blending and compression), dry granulation or compaction (densifying the drug or drug/powder blend through slugging or with a compactor to obtain flowable, compressible granules), fat-wax mot melt granulation (embedding of drug in molten fatty alcohols, followed by cooling, sizing, blending and compression), and film-coating of particulates (dry blend, wet granulation, kneading, extrusion, spheronization, drying, film-coating, followed by blending of differing; species of film-coated spheres, and compression).

Cellulose derivatives capable of achieving controlled drug release include the esters: cellulose acetates, cellulose acetate propionates, and cellulose acetate burtyrates, all of which are readily available. A widely used polymer for retarding dissolution of a drug for oral dosage is ethyl cellulose, a cellulose derivative in which part of the hydroxyl sites are substituted with ethoxyl groups. It is available in various grades, whose properties are dependant upon the degree of ethoxyl substitution and the molecular weight. For example, commercial grades are available with substitution of 2.25 to 2.60 ethoxyl units per anhydroglucose unit (44% to 50% ethoxyl content).

For parenteral use, it is desirable to use a polymer that is biocompatible and bioerodible. Parenteral dosage forms may use polylactic acid and copolymers with polyglycolic acid, which are hydrolytically degraded in the body to hydroxycarboxylic acids and eventually metabolized to carbon dioxide and water.

In preferred embodiments, the controlled-release matrix comprises a cellulose polymer, such as cellulose ether. Examples of cellulose ethers include methyl cellulose (MC), methylhydroxyethyl cellulose (MHEC or HEMC), methylhydroxypropyl cellulose (MHPC or HPMC), hydroxyethyl cellulose (HEC), ethyl-HEC, methyl-ethyl-HEC (MEHEC), butylglycidylether-HEC, laurylglycidylether-HEC, carboxymethylated MHEC or MHPC, and sodium carboxymethyl cellulose (Na-CMC). Derivatives of HEC with alkyl side-chains containing more than two carbon atoms are referred to as hydrophobic modified HEC (hmHEC or HMHEC).

An especially suitable group of polymers is the polymers sold under the Trade Mark Methocel. Furthermore, the Methocel used may be Methocel K4M, K15M, K100M, or E, J, F grades depending on the release characteristics desired.

Hydroxypropylmethyl cellulose is commercially available in various grades, under several tradenames, including METHOCEL, E, F, J and K from The Dow Chemical Co., USA, HPM from British Celanese Ltd. England and Metaluse SH from Shin Etsu, Ltd, Japan. The various grades available under a given tradename represent differences in methoxyl and hydroxypropoxyl content as well as molecular weight. The methoxyl content ranges from 16.5 to 30 weight % and the hydroxypropoxyl content ranges from 0 to 32 weight %, as determined by the method described in ASTM D-2363-72. All of these various forms of hydroxypropylmethyl cellulose are contemplated to be used in the present invention. For example, the present invention contemplates the use of Methocel K in its various forms having a methoxyl content of 19-24% and a hydroxypropoxyl content of 7-12%, Methocel E in its various forms, having a methoxyl content of 28-30 to and a hydroxypropyl content of 7-12%, Methocel F in its various forms having a methoxyl content of 27-30% and a hydroxypropoxyl content of 4-7.5%, Methocel A in its various forms, having a methoxyl content of 27.5-31.5% and about 0% hydroxypropoxyl content.

Commercial designations of the various hydroxypropylmethyl cellulose are based on the viscosities of 2% aqueous solutions at 20 degrees C. The viscosities range from 15 cps to 30,000 cps and represent number average molecular weights ranging from about 10,000 to over 150,000, as calculated from the data in the “Handbook of Methocel Cellulose Ether Products” (The Dow Chemical Co., 1974).

Examples of hydroxypropylmethyl cellulose include Metalose 60 5H50 which is a hydroxypropylmethyl cellulose having a hydroxypropoxyl content of 9-12 weight % and a number average molecular weight of less than 50,000; Methocel E4M, having a 28-30 weight % methoxyl content, a viscosity of 4000 cps, a hydroxy-propoxyl weight % of 7-12 and a number average molecular weight of 93,000; Methocel E10M, having a viscosity of 10,000 cps, a 28-30 weight % methoxyl content, 7-12 weight % hydroxypropoxyl, Methocel K4M, having a number average molecular weight of 89,000, viscosity of 4,000, 19-24% weight % methoxyl content, and a 7-12 weight % hydroxypropoxyl content; Methocel K15M, having a number average molecular weight of 124,000, a 19-24 weight % methoxyl content and a 7-12 weight % hydroxypropoxyl content; and K100M, having a viscosity of 100,000 cps and a 19-24 weight % methoxyl content and is 7-12 weight % hydroxypropoxyl content, Methocel J5M, J12M, J20M and J75M, having viscosities of 5,000, 12,000, 20,000, and 75,000, cps, respectively and the like. Various hydroxypropylmethyl cellulose materials which can also be used in the present formulation are described in U.S. Pat. No. 3,870,790 to Schorr, U.S. Pat. No. 4,226,849 to Schorr, U.S. Pat. No. 4,357,469 to Schorr, U.S. Pat. No. 4,369,172 to Schorr, et al., U.S. Pat. No. 4,389,393 to Schorr, et al., U.S. Pat. No. 4,259,314 to Lowey, U.S. Pat. No. 4,540,566 to Davis, et al., U.S. Pat. No. 4,556,678 to Hsiau, the contents of all of which are incorporated herein by reference. The present formulation may contain one cellulose ether or a combination of cellulose ethers.

If one wishes to delay release of the active ingredient (anabaseine compound(s)) in vivo in capsule or tablet form, a combination of water soluble and a water insoluble polymer or a mixture of such polymers can be used, with the ratio of the water soluble to water insoluble polymer being varied to give the desired rate of release.

The excipients commonly used in the formulations are as follows: Microcrystalline cellulose (MCC; commercially available from Avicel), lactose, Mannitol and Di-Pac (compressible sugar) as diluents; magnesium aluminum silicate, xanthan gum, polyvinylpyrrolidone and cellulose compounds as anti-clumping agents; starch hydroxypropyl methylcellulose (HPMC E-10) and xanthan gum as binders; sweetening agents such as sucrose, saccharin sodium, Sucralose and Magnasweet; calcium phosphate as hardness enhancer; talc as a glidant and magnesium stearate as a lubricant. Active ingredients not present as tannate salt complexes also can be included in the formulation.

MCC is particularly useful for direct compression. It is available in a variety of grades, differing in parameters such as mean particle size, particle size distribution, density, and moisture. Materials may be utilized that are composed of MCC co-processed with other excipients, providing a range of flow properties and compressibilities for MCC products that result in differing tablet strengths and manufacturing constraints, which potentially have a bearing on drug dissolution.

CR tablets may be made by incorporating the anabaseine compound within a matrix system, including but not limited to: a hydrophilic matrix system, a hydrophobic (plastic matrix system), a hydrophilic/hydrophobic matrix system, a fat/wax system, and a film-coated particulate system.

Hydrophilic matrix systems show uniform and constant drug diffusion from a tablet prepared with a hydrophilic, gelling excipient after it is placed in an aqueous environment. Drug release is controlled by a gel diffusional barrier which is formed. The process is usually a combination of gel hydration, drug diffusion, and gel erosion.

Hydrophobic (plastic) matrix systems utilize inert, insoluble polymers and copolymers to form a porous skeletal structure in which the drug is embedded. Controlled drug release is affected by diffusion of drug through the capillary wetting channels and pores of the matrix, and by erosion of the matrix itself.

Hydrophilic/hydrophobic matrix systems utilize a combination of hydrophilic and hydrophobic polymers that forms a soluble/insoluble matrix in which the drug is embedded. Drug release is by pore and gel diffusion as well as tablet matrix erosion. The hydrophilic polymer is expected to delay the rate of gel diffusion.

In fat-wax matrix systems, the drug is incorporated in a hot melt of a fat-wax matrix, solidified sized and compressed with appropriate tablet ingredients. Controlled-release of the drug is affected by pore diffusion and erosion of the fat-wax matrix. The addition of a surfactant as a wicking agent helps water penetration of the matrix to cause erosion.

Film-coated particulate systems include time-release granulations, prepared by extrusion-spheronization process or by conventional granulation process that have been film-coated to produce differing species of controlled-release particles with specific drug release characteristics.

Controlled-release particles may be compressed together with appropriate tabletting excipients to produce tablets with the desired controlled-release profile. In this context, drug release is by particle erosion in either acid (gastric) or alkaline (intestinal) pH.

In one aspect of the invention, the composition is suitable for oral administration for extended-release of one or more anabaseine compounds, and the composition comprises: a) from about 0.1% to about 80% by weight anabaseine compound(s); and from about 99.9% to about 20% by weight extended-release agent. The composition may provide extended-release of the one or more anabaseine compounds over a period of from about 2 hours to 48 hours. In some embodiments, extended-release of the one or more anabaseine compounds over a period of at least 4 to 8 hours is achieved. In some embodiments, extended-release of the one or more anabaseine compounds over a period of at least 6 to 8 hours is achieved. Preferably, the amount of anabaseine compound concentration that is released in an extended fashion over a period of time (e.g., 2 hours to 48 hours, 4 hours to 8 hours, 6 hours to 8 hours, etc.) is a clinically effective (therapeutically effective and/or prophylactically effective) amount. In some embodiments, the extended-release agent comprises a hydrophilic polymer, such as cellulose ether. The composition may further include one or more of the following additional ingredients: lubricant, filler, binder, disintegrant, lubricants, auxiliary excipients such as glidants and solubilizers. It should be understood that the aforementioned ranges include all units and sub-ranges encompassed by the range.

### Therapeutic and Prophylactic Methods

The controlled-release formulation containing one or more anabaseine compounds can be administered enterally, parenterally, or by gradual perfusion over time. The formulation can be administered intravenously (IV), intraperitoneally, intramuscularly, subcutaneously, intracavity, transdermally, or orally. In preferred embodiments, the controlled release formulation is administered orally, intranasally, sublingually, or by inhalation. In particularly preferred embodiments, the controlled-release formulation is an orally administered formulation.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose, and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. In order to form a pharmaceutically acceptable composition suitable for effective administration, such compositions will contain an effective amount of the nicotinic receptor agent, together with a suitable amount of a carrier vehicle.

In one aspect, the invention includes a method of maintaining a serum or plasma anabaseine compound concentration in a subject for a duration of from about 4 hours to about 8 hours, the method comprising administering an effective amount of a controlled-release composition described herein. In some embodiments, a serum or plasma anabaseine compound concentration in a subject for a duration of from about 6 hours to about 8 hours is achieved. Preferably, the serum or plasma anabaseine compound concentration maintained in the subject is a clinically effective (therapeutically effective and/or prophylactically effective) amount.

In another aspect of the invention, there is provided a method of maintaining a concentration of an anabaseine compound in the brain of a subject for a duration of from about 4 hours to about 8 hours, the method comprising administering an effective amount of a controlled-release composition described herein. In some embodiments, an anabaseine compound concentration is achieved in the brain for a duration of from about 6 hours to about 8 hours. Preferably, the anabaseine compound concentration maintained in the brain is a clinically effective (therapeutically effective and/or prophylactically effective) amount.

In another aspect, the invention includes a use of a controlled-release composition described herein for preparation of a medicament for maintaining a serum concentration of an anabaseine compound or active metabolite thereof in a subject for a duration of from about 4 hours to about 8 hours. In another aspect of the invention, there is provided a use of a controlled-release composition described herein for preparation of a medicament for maintaining a concentration of an anabaseine compound or active metabolite thereof in the brain of a subject for a duration of from about 4 hours to about 8 hours. In another aspect of the invention, there is provided a use of a controlled-release composition described herein for preparation of a medicament for treatment of a disease or condition caused or exacerbated by a defect in, and/or malfunctioning of nAChR.

### Dosage Forms and Dosage Amounts and Dosage Frequency

In general, the amount of the anabaseine compound or compounds present in a composition depends inter alia on the specific anabaseine compound and formulation, the age and condition of the subject, and the disease or conditions to be treated and/or prevented, the route of administration, and the dosage frequency.

The dosage frequency also depends on the disease or condition to be treated and/or prevented, amount or concentration of the anabaseine compound(s), the specific composition used, the route of administration, and may incorporate subject-specific variation including, but not limited to age, weight, gender, genetic background, and overall health. For example, a nasal formulation may be administered once daily, e.g., in order to achieve a relatively fast onset of the therapeutic effect, or it may be administered more often. The same criteria for selecting dosage frequency applies to other dosage forms including but not limited to a plain tablet composition, a buccal composition, a rectal composition, an oral composition, a topical composition, an ocular composition, or other compositions.

Typically, the anabaseine compounds described herein are formulated for use in humans. Anabaseine compounds can also include veterinary formulations, e.g., pharmaceutical preparations suitable for veterinary uses, e.g., for the treatment of livestock or domestic animals, e.g., dogs, cats, racehorses, etc. It has also been reported that anabaseine compounds may be insecticidal (Sultana et al., 2002, Insect, Biochem. Mol. Biol., 32:637-643). Accordingly, insecticidal anabaseine compounds can be formulated to enhance penetration into the insect nervous system and duration of action.

Actual dosage levels of the anabaseine compound or compounds in the controlled-release formulations described herein may be varied so as to obtain an amount of the anabaseine compound(s) which is effective to achieve the desired therapeutic effect for a particular subject, without being toxic to the subject.

The selected dosage level will depend upon a variety of factors including but not limited to the activity of the anabaseine compound (or the ester, salt, amide or formulation thereof); the route of administration; the time of administration; the rate of excretion of the particular anabaseine compound being employed; the duration of the treatment; other drugs, compounds and/or materials used in combination with the anabaseine compound; the age, sex, weight, condition, general health and prior medical history of the subject being treated; and like factors well known in the medical arts.

A physician or veterinarian having ordinary skill in the art can readily determine and prescribe the effective amount of the pharmaceutical composition required. For example, the physician or veterinarian could start doses of the anabaseine compound at levels lower than that required in order to achieve the desired therapeutic effect and gradually increase the dosage until the desired therapeutic effect is achieved.

In general, a suitable dose of an anabaseine compound or compounds will be the lowest dose effective to produce a therapeutic effect. Such an effective dose will generally depend upon the factors described above. Preferred formulations include oral and intravenous forms (IV), nasal forms, sublingual and metered dose inhaler forms. Generally, intravenous and oral forms of the anabaseine compound for a subject will range from about 0.1 to about 50 mg per kilogram of body weight per day.

Intranasal formulations and patch formulations may be used. Generally, intranasal formulations and patch formulations of the anabaseine compound for a subject will range from about 0.01 to about 10 mg per kilogram of body weight per day.

The effective dose of the anabaseine compound may be administered as two, three, four, five, six or more sub-doses administered separately at appropriate intervals throughout the day, optionally, in unit dosage forms.

The subject receiving this treatment is any animal in need, including primates, in particular humans, and other mammals such as equines, cattle, swine and sheep; and poultry and pets such as dogs and cats among others in general.

The anabaseine compound may be administered to a subject by any route capable of delivering a therapeutically effective amount of the compound including but not limited to administration by oral, parenteral, intracranial, intraorbital, intracapsular, intraspinal, intracisternal, intrapulmonary, intravenous, intramuscular, intra-arterial, intramedullary, intrathecal, intraventricular, transdermal, subcutaneous, intraperitoneal, intranasal, enteral, topical, sublingual, buccal, gingival, palatal or rectal means.

Typically, the anabaseine compound is given in forms suitable for each administration route. For example, the anabaseine compound may be administered parenterally by injection, infusion or inhalation; administered topically by lotion or ointment; or administered rectally by suppositories. Typical forms of administration described herein are not intended to be either limiting or exhaustive, but merely illustrative.

The phrases “parenteral administration” and “administered parenterally” as used herein mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection and infusion.

The phrases “systemic administration,” or “administered systemically,” as used herein mean the administration of a compound, drug or other material such as an anabaseine compound so that it enters the subject's system by a direct route or parenteral route and thus is subject to metabolism and other like processes (for example, by subcutaneous administration). The phrases “peripheral administration” and “administered peripherally,” as used herein, mean the administration of a compound, drug or other material such as an anabaseine compound so that it enters the subject's system by an indirect or localized route and thus is subject to metabolism and other like processes (for example, by topical administration).

Regardless of the route of administration, the anabaseine compounds described herein can be formulated into pharmaceutically acceptable dosage forms such as described, or other dosage forms known to those of skill in the art.

The phrase “pharmaceutically acceptable” as used herein can refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The anabaseine compounds can be administered alone or in admixtures with pharmaceutically acceptable and/or sterile carriers and can also be administered in conjunction with other drugs (e.g., other cardiovascular agents, antimicrobial agents, antipsychotic agents, etc.). Multiple routes of simultaneous or sequential administration (e.g., oral and transdermal) are also contemplated.

Controlled-release formulations of anabaseine compounds can be formulated in any manner suitable for a desired delivery route. Typically, formulations include all physiologically acceptable compositions. Such formulations include one or more anabaseine compounds and a controlled-release agent in combination with any physiologically acceptable carrier or carriers. The formulation may also enhance, alter, or modify the effect or the anabaseine compound and/or physiological milieu of the anabaseine compound.

The anabaseine compounds may be formulated for administration in any way for use in human or veterinary medicine. The anabaseine compound may be active itself, or may be a prodrug, e.g., capable of being converted to an active compound in a physiological setting.

Anabaseine compounds as described herein may be formulated for administration with any biologically acceptable medium, including but not limited to water, buffered saline, polyol (for example, glycerol, propylene glycol, liquid polyethylene glycol and the like) or suitable mixtures thereof. The optimum concentration of the anabaseine compound in the chosen medium can be determined empirically, according to procedures well known in the art. As used herein, “biologically acceptable medium” includes any and all solvents, dispersion media, and the like which may be appropriate for the desired route of administration of the pharmaceutical preparation. The use of a biologically acceptable medium for pharmaceutically active substances is known in the art. Suitable biologically acceptable media and their formulation are described, for example, in the most recent version of Remington's Pharmaceutical Sciences (Remington's Pharmaceutical Sciences. Mack Publishing Company, Easton, Pa., USA 1985).

The controlled-release formulations may contain suitable physiologically acceptable carriers comprising excipients and/or auxiliaries which facilitate processing of the anabaseine compounds into preparations which can be used pharmaceutically. Controlled-release formulations of the anabaseine compounds may also include additional agents which increase or otherwise affect the bioavailability of the drug. As used herein, “bioavailability” refers to the effect, availability and persistence of the anabaseine after being administered to a subject.

Pharmaceutically acceptable carriers can be any pharmaceutically acceptable material, composition, or vehicle, including but not limited to a liquid or solid filler, diluent, excipient, solvent or encapsulating material, involved in carrying or transporting the subject agonists to an organ, or portion of the body. Each carrier must be compatible with the other ingredients of the formulation and not injurious to the subject. Some examples of materials which can serve as pharmaceutically acceptable carriers include but are not limited to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

The anabaseine compound may be capable of forming pharmaceutically acceptable salts such as inorganic and organic acid or base addition salts of the anabaseine compounds described herein (See, for example, Berge et al., 1977, “Pharmaceutical Salts”, J. Pharm. Sci., 66:1-19), and may be used in the dosage forms and methods of the invention. In particular, HCl salts of the anabaseine compounds may be used. Other salt forms include hydrobromide, hydroiodide, bisulphate, acid citrate, bitartrate, ethanesulphonate, sulphate, phosphate or acid phosphate, acetate, maleate, fumarate, lactate, tartrate, L-tartrate, citrate, gluconate, benzenesulphonate (besylate), p-toluenesulphonate (tosylate), methanesulphonate (mesylate), esylate, succinate, salicylate, nitrate, sulfate, etc.

Formulations of the anabaseine compounds can also include wetting agents; emulsifiers and lubricants such as sodium lauryl sulfate and magnesium stearate; coloring agents; release agents; coating agents; sweetening, flavoring, and/or perfuming agents; preservatives; and antioxidants.

Examples of pharmaceutically acceptable antioxidants include but are not limited to water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Controlled-release formulations of anabaseine compounds may also incorporate buffering agents and/or salts to aid absorption or stabilize the anabaseine compound. Other additives, such as chelating agents, enzymatic inhibitors, and the like, which would facilitate the biological activity of the pharmaceutical composition may also be incorporated in the formulation. Formulations of anabaseine compounds may also contain opacifying agents.

The controlled-release formulations of anabaseine compounds may be presented in unit dosage form and may be prepared by any methods known in the art. The amount of anabaseine compound that can be combined with a carrier material to produce a single dosage form may vary. For example, the amount of anabaseine compound in a given formulation may depend upon the subject being treated and/or the particular mode of administration. The amount of anabaseine compound which can be combined with a carrier to produce a single dosage form will generally be that amount of the anabaseine compound which produces a therapeutic effect.

Methods of preparing these formulations include the step of bringing into association an anabaseine compound with the carrier and/or one or more accessory ingredients. Some formulations may be prepared by bringing an anabaseine compound in association with liquid carriers, finely divided solid carriers, or both, and then shaping the product.

Formulations of the anabaseine compound suitable for oral administration may be in the form of a solid (capsules, cachets, pills, tablets, lozenges, powders, dragees, granules); or as a solution or a suspension in an aqueous or non-aqueous liquid; or as an oil-in-water or water-in-oil liquid emulsion; or as an elixir or syrup; or as pastilles (using an inert base, such as gelatin and glycerin, or sucrose and acacia); and/or as mouth rinses or washes and the like; or as a bolus, electuary or paste.

Solid controlled-release formulations of anabaseine compounds may have pharmaceutically acceptable carriers and extenders including but not limited to sodium citrate or dicalcium phosphate; starches; lactose; sucrose; glucose; mannitol; and/or silicic acid. Solid formulations of the anabaseine compound can include additional components including but not limited to binders such as carboxymethylcellulose, alginates, gelatin, polyvinyl pyrrolidone, sucrose and/or acacia; humectants such as glycerol; disintegrating agents such as agar-agar, calcium carbonate, potato or tapioca starch, alginic acid, certain silicates, and sodium carbonate; solution retarding agents such as paraffin; absorption accelerators such as quaternary ammonium compounds; wetting agents such as cetyl alcohol and glycerol monostearate; absorbents such as kaolin and bentonite clay; lubricants such a talc, calcium stearate, magnesium stearate, solid polyethylene glycols, sodium lauryl sulfate, and mixtures thereof; and coloring agents. The formulation may also include buffering agents, particularly when the anabaseine compound is in the form of a capsule, tablet or pill.

Solid formulations may also include fillers for soft and hard-filled gelatin capsules using excipients such as lactose or milk sugars, as well as high molecular weight polyethylene glycols and the like.

Solid formulations such as pills and tablets may be formed by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of powdered anabaseine compound moistened with an inert liquid diluent.

Solid formulations of anabaseine compounds described herein, such as dragees, capsules, pills and granules, may optionally be scored or prepared with coatings and shells, such as enteric coatings and other coatings. Solid dosage forms may be formulated so as to provide slow or controlled-release of the anabaseine compound, as described herein. Thus, solid formulations could include any material that could provide a desired release profile of the anabaseine compound, including but not limited to hydroxypropylmethyl cellulose in varying proportions, or other polymer matrices, liposomes and/or microspheres.

Formulations of anabaseine compounds may also be formulated to release the anabaseine compound only, or preferentially, in a certain portion of the gastrointestinal tract, for example, by including an embedding agent. Examples of embedding agents which can be used include but are not limited to polymeric substances and waxes. The anabaseine compound may also be in microencapsulated form, if appropriate, with one or more of the above-described excipients.

Coated or encapsulating formulations of anabaseine compounds may also be formulated to deliver pulsatile, sustained, or extended-release. For example, one method of pulsatile release can be achieved by layering multiple coatings of anabaseine compound, or by incorporating the anabaseine compound within different regions of the formulation having different release times.

Liquid dosage formulations for oral administration of the anabaseine compounds may include pharmaceutically acceptable emulsions, microemulsions, solutions, suspensions, syrups and elixirs. In addition to the anabaseine compound, the liquid dosage formulations may contain inert diluents commonly used in the art, including but not limited to water or other solvents; solubilizing agents and emulsifiers, such as ethyl alcohol, isopropyl alcohol, ethyl carbonate, ethyl acetate, benzyl alcohol, benzyl benzoate, propylene glycol, 1,3-butylene glycol; oils (in particular, cottonseed, groundnut, corn, germ, olive, castor and sesame oils); glycerol; tetrahydrofuryl alcohol; polyethylene glycols; and fatty acid esters of sorbitan, and mixtures thereof.

The anabaseine compound may also be formulated as a suspension. Suspensions of the anabaseine compound may include suspending agents. Examples of suspending agents include but are not limited to ethoxylated isostearyl alcohols; polyoxyethylene sorbitol and sorbitan esters; microcrystalline cellulose; aluminum metahydroxide; bentonite; agar-agar; tragacanth; and mixtures thereof.

Formulations of the anabaseine compound for rectal or vaginal administration may be presented as a suppository. Suppository formulations may be prepared by mixing one or more anabaseine compounds with one or more suitable nonirritating excipients or carriers. Suitable carriers include any compound which is solid at room temperature but liquid at body temperature, and therefore will melt in the rectum or vaginal cavity and release the anabaseine compound. Examples of such carriers include but are not limited to cocoa butter; polyethylene glycol; a suppository wax or a salicylate.

Formulations of the anabaseine compound suitable for vaginal administration also include pessaries, tampons, creams, gels, pastes, foams or spray formulations containing such carriers as are known in the art.

Formulations of the anabaseine compound suitable for the topical or transdermal administration include powders, sprays, ointments, pastes, creams, lotions, gels, solutions, patches and inhalants. The anabaseine compound may be mixed under sterile conditions with a pharmaceutically acceptable carrier, and with any preservatives, buffers, or propellants which may be required.

Powders and sprays may contain, in addition to an anabaseine compound, excipients such as lactose, talc, silicic acid, aluminum hydroxide, calcium silicates and polyamide powder, or mixtures of these substances. Sprays may contain customary propellants, such as chlorofluorohydrocarbons and volatile unsubstituted hydrocarbons, such as butane and propane.

The anabaseine compound may also be formulated as a transdermal patch. Transdermal patches have the added advantage of providing controlled delivery of the anabaseine compound into the body. Such formulations may be made by dissolving or dispersing the anabaseine compound in the proper medium. Absorption enhancers may also be used to increase the flux of the compound across the skin. The rate of flux may be controlled. Examples of ways of controlling the rate of flux include but are not limited to rate controlling membranes or dispersing the compound in a polymer matrix or gel.

Ophthalmic formulations of the anabaseine compound include, but are not limited to, eye ointments, powders, solutions and the like.

Formulations of anabaseine compounds for parenteral administration may have one or more anabaseine compounds in combination with one or more pharmaceutically acceptable isotonic aqueous or nonaqueous solutions, dispersions, suspensions or emulsions, or powders which may be reconstituted into sterile injectable solutions or dispersions just prior to use. Parenteral formulations may contain antioxidants; buffers or solutes which render the formulation isotonic with the blood of the intended subject; bacteriostats; suspending; or thickening agents.

Injectable depot formulations of the anabaseine compound can be made by forming microencapsulated matrices of the anabaseine compounds in biodegradable polymers. Examples of biodegradable polymers include, but are not limited to polylactide-polyglycolide, poly(orthoesters) and poly(anhydrides). The ratio of anabaseine compound to polymer and the nature of the particular polymer employed can affect the rate of anabaseine compound released. Depot injectable formulations can also be prepared by entrapping the drug in liposomes or micro emulsions.

Proper fluidity of liquid, suspension and other formulations of the anabaseine compounds can be maintained by the use of coating materials such as lecithin; by the maintenance of the required particle size in the case of dispersions; or by the use of surfactants.

Formulations of the anabaseine compounds may also include anti-contamination agents for the prevention of microorganism contamination. Anti-contamination agents may include but are not limited to antibacterial and antifungal agents, for example, paraben, chlorobutanol, phenol sorbic acid, and the like.

Formulations of the anabaseine compound may also be sterilized by, for example, by filtration through a bacteria-retaining filter, or by incorporating sterilizing agents in the form of sterile solid formulations which can be dissolved in sterile water, or some other sterile medium immediately before use or formulation.

Formulations of the anabaseine compounds may also include isotonic agents such as sugars, sodium chloride, and the like.

The present formulation also contains optional components. For example, although not necessary, in a preferred embodiment, the present formulation additionally contains a lubricant that is typically used in the pharmaceutical arts for oral tablets. As used herein, the term “lubricant” refers to a material which can reduce the friction between the die walls and the punch faces which occurs during the compression and ejection of a tablet. The lubricant prevents sticking of the tablet material to the punch faces and the die walls. As used herein, the term “lubricant” includes anti-adherents. Examples of lubricants include stearate salts, e.g., alkaline earth, and transition metal salts thereof, e.g., calcium, magnesium, or zinc; stearic acid, polyethylene oxide, talc, hydrogenated vegetable oil, and vegetable oil derivatives, silica, silicones, high molecular weight polyalkylene glycol, e.g., high molecular weight polyethylene glycol, monoesters of propylene glycol, saturated fatty acid containing about 8-22 carbon atoms and preferably 16-20 carbon atoms. The preferred lubricants are the stearate salts, stearic acid, talc and the like.

To avoid tablet sticking during formation and or ejection, the present formulation contemplates utilizing a lubricating effective amount of the lubricant. Preferably, the lubricant is present in amounts ranging from about 0.1% to about 5% by weight and more preferably from about 1% to about 4% by weight of the tablet.

Another optional ingredient is an inert filler. The filler may substantially water soluble or water insoluble. A filler can be used if needed or desired, although it is not necessary for the present formulation. The fillers used in the present formulation are preferably those typically used in the pharmaceutical arts for oral tablets. Examples include calcium salts, such as calcium sulfate, dicalcium phosphate, tricalcium phosphate, calcium lactate, calcium gluconate, and the like, glycerol phosphate; citrates; and mixture thereof, and the like. In some embodiments, the inert filler of the controlled-release formulation of the present invention comprises a pharmaceutically acceptable saccharide, including a monosaccharide, a disaccharide, or a polyhydric alcohol and/or mixtures of any of the foregoing. Examples thereof include sucrose, dextrose, lactose, microcrystalline cellulose, fructose, xylitol, sorbitol, mixtures thereof and the like. The filler, if present, is present in amounts ranging from about 1% to about 90% by weight.

Other optional ingredients that are also typically used in pharmaceuticals may also be present, such as coloring agents, preservatives (e.g., methyl parabens), artificial sweeteners, flavorants, antioxidizing agents and the like. Artificial sweeteners include, but are not limited to saccharin sodium, aspartame, dipotassium glycyrrhizinate, stevia, thaumatin and the like. Flavorants include, but are not limited to lemon, lime, orange and menthol. The colorants include, but are not limited to various food colors, e.g., FD & C colors, such as FD & C Yellow No. 6, food lakes and the like. Examples of anti-oxidants include ascorbic acid, sodium metabisulphite, and the like. These optional ingredients, if present, are preferably present in amounts ranging from about 0.1% to about 5% by weight of the tablet and most preferably less than about 3% (w/w) of the tablet.

The present formulation of the present invention is prepared by blending the anabaseine compound(s) with the lubricant, cellulose ether, hydrocolloid, and the other optional ingredients. The ingredients can be mixed in a typical blender that is normally utilized in the pharmaceutical arts, such as a Hobart mixer, V-blender, a planetary mixer, Twin shell blender and the like. The ingredients are blended together typically at about ambient temperature; no additional heating is necessary, although slight modifications of temperature therefrom could be utilized. It is preferred that the blending be conducted at temperatures ranging from about 10 degrees C. to about 45 degrees C.

The ingredients in the formulation are preferably mixed together in a large batch using techniques well known in the pharmaceutical arts and are intimately intermixed until the mixture is homogenous with respect to the drug.

The term “homogenous” with respect to the drug is used to denote that the various components are substantially uniform throughout the invention, i.e., a substantially homogeneous blend is formed.

When the mixture is homogeneous, a unit dosage amount of the mixture can be compressed into a tablet form using a tablet machine typically utilized in the pharmaceutical arts. More specifically, the mixture is fed to the die of a tablet press and sufficient pressure is applied to form a solid tablet. Such pressure can vary, and typically ranges from about 1,000 psi to about 6,000 psi and preferably about 2,000 psi force. The solid formulation according to the present invention is compressed to a sufficient hardness to prevent the premature ingress of the aqueous medium into the tablet. Preferably, the formulation is compressed into a tablet form which is of the order of 5-20 Kp and more preferably 8-20 Kp as determined by a Schleuniger hardness tested.

In a variation, all of the above steps are repeated, except that the mixing is initially performed in the absence of a lubricant. When the mixture is homogeneous with respect to the drug, then the lubricant is added and the mixing is continued until the lubricant is substantially evenly dispersed in the mixture. Then the mixing is terminated, and the mixture is immediately thereafter compressed into a tablet, as described hereinabove.

Another procedure for preparing the formulation of the present invention is by the wet granulation process in which all of the components except the lubricant are mixed with a sufficient amount of a granulating solvent to form a substantially uniform blend. The granulating vehicle is one that is inert with the components and has a low boiling point, i.e., preferably less than about 120 degrees C. It is preferably a solvent that contains OH groups, such as an alcohol containing 1-4 carbon atoms, e.g., isopropyl alcohol or ethanol or water and the like. An aqueous dispersion can also be utilized. In a preferred embodiment, the type of granulating vehicle used is dependent upon the identity of the sustained-release polymer. For example, it is preferred that when hydroxypropylmethyl cellulose is utilized, the granulating vehicle is water or alcohol.

The substantially uniformly blended mixture may optionally be milled, e.g., passed through a screen, sieve, etc. to reduce the size of the particles thereof. The screen or sieve, and the like is preferably less than about 140 mesh, and more preferably less than about 100 mesh, and even more preferably, less than about 40 mesh, and most preferably less than about 20 mesh.

Next, the blend is dried. In this step, the solvent is removed from the blend by physical means known to the skilled artisan, e.g., by evaporation. The resulting granules are again milled, e.g., passed through a screen or sieve to further reduce the size of the particles to the desired size. Then the lubricant is added, and the granules are mixed to provide a uniform blend, i.e., homogenous with respect to the drug and then the resulting mixture is compressed to form a tablet. In a preferred variation, the blend can be simultaneously granulated in the granulation vehicle and dried such as using a fluid bed granulation process.

After the tablet is formed, the tablet can be coated with materials normally used in pharmaceuticals, if desired. If coated, the coating is prepared by techniques known in the art. However, the formulation of the present invention is preferably uncoated.

The tablet product is obtained which has the desired hardness and friability typically found for phaimaceutical tablets. The hardness is preferably 5-25 Kp and more preferably 8-20 Kp.

For oral dosage forms, is intended that the anabaseine compound be released slowly or according to a prescribed rate after ingestion within the body as the formulation progresses along the gastro-intestinal tract. In this regard, the gastro-intestinal tract is considered to be the abdominal portion of the alimentary canal, i.e., the lower end of the esophagus, the stomach and the intestines.

The dosages of a formulation according to the invention correspond to the normal dosages of the particular active ingredient known to the skilled artisan. The precise amount of drug administered to a patient will depend on a number of factors, including the age of the patient, the severity of the condition and the past medical history, among other factors, and lie within the discretion of the administering physician. For guideline as a suitable dosage, reference is made to the Physicians Desk Reference.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### Preparation of a Controlled-Release Capsule Dosage Form of DMXBA

Controlled-release (CR) capsules were prepared using 30% K100M METHOCEL (Dow Chemical Co.) to total weight of DMXBA prepared as the dihydrochloride salt. Each capsule contained a dose of 150 mg of DMXBA. Avicel-PH102 (Microcrystalline Cellulose; FMC Corp., Newark, Del.) was used as the filler. At time zero each capsule (suspended in a nylon net) was immersed near the top center of a beaker containing 1.0 liter of simulated gastric fluid (This contained Sigma porcine pepsin 3.2 grams, 0.9% sodium chloride and HCl to obtain pH 1.20) that had been pre-equilibrated at 37±0.5 degrees C. A magnetic stir bar was used to slowly agitate the fluid at 100 rpm. These conditions were recommended in the USP for such tests of controlled-release. At nine different times over an almost 24 hr period a 1.00 ml sample was removed and then replaced by an equal volume of the simulated gastric fluid. These samples were kept frozen (and in the dark) before they were analyzed by spectrophotometry and by HPLC.

Table 1 lists the release characteristics for four capsules produced and evaluated in vitro in identical fashion. Spectrophotometric absorbance readings were taken after 1.0 ml of each sample was diluted by adding 2.0 ml of water containing HCl to obtain pH 1.2 (as in the actual dissolution medium containing pepsin, NaCl, and HCl, pH 1.2, simulate gastric fluid).

TABLE 1

| Time, | Abs, | Abs, | Abs, | Abs, | | |
|---|---|---|---|---|---|---|
| hours | 450 nm | 450 nm | 450 nm | 450 nm | Mean | S.E.M. |
| 0 | 0 | 0 | 0 | 0 | 0 | 0 |
| 0.5 | 0.11 | 0.113 | 0.135 | 0.154 | 0.128 | 0.01 |
| 1 | 0.281 | 0.27 | 0.298 | 0.293 | 0.285 | 0.006 |
| 2 | 0.485 | 0.529 | 0.509 | 0.472 | 0.499 | 0.013 |
| 3 | 0.617 | 0.693 | 0.618 | 0.615 | 0.636 | 0.019 |
| 4 | 0.732 | 0.795 | 0.696 | 0.708 | 0.733 | 0.022 |
| 5.5 | 0.87 | 0.976 | 0.841 | 0.817 | 0.876 | 0.035 |
| 7 | | 1.057 | 0.92 | 0.865 | 0.947 | 0.057 |
| 9.5 | 1.0515 | 1.169 | 1.069 | 0.98 | 1.067 | 0.039 |
| 23.5 | 1.059 | 1.234 | 1.157 | 1.183 | 1.158 | 0.037 |

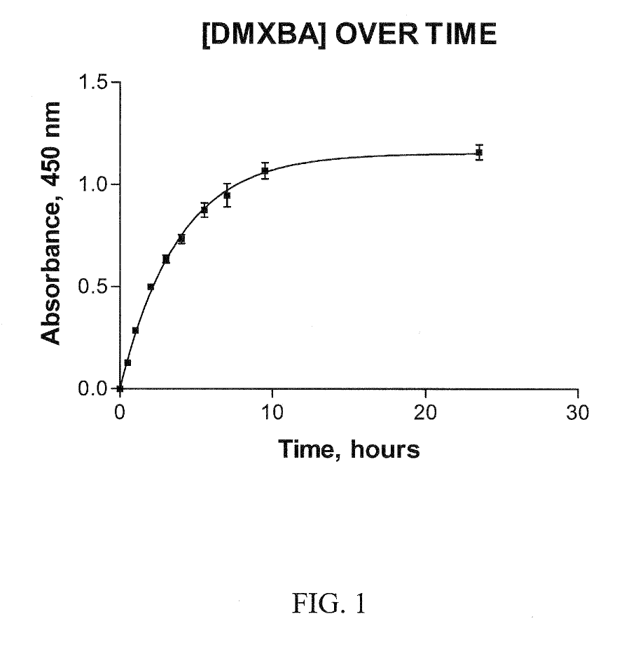

The absorbance at 450 nm is for a 1:3 dilution of the fluid, diluted with HCl solution of pH 1.2 (as for the dissolution fluid). The dilution was necessary in order to accurately measure the absorbance; the undiluted fluid sample absorbance was too high to measure accurately. FIG. 1 shows the release profile of Table 1.

On the average, the DMXBA released was 134 mg of the 150 mg in a CR capsule (89% recovered), without correction for the DMXBA removed during multiple sampling. HPLC was conducted on each of the four final samples of the capsule dissolution medium. These analytical separations confirmed that the DMXBA did not change during the release process or after formulation with the CR material.

Two non-extended-release (immediate release) capsules of 150 mg of DMXBA were also prepared, which showed a very rapid increase in DMXBA relative to the CR form of the same dose, measured under the same conditions. As shown in Table 2, dissolution was essentially complete within 0.5 hr or less when the CR material was not present.

TABLE 2
Dissolution of 150 mg DMXBA capsule lacking CR material

| Time, | Abs, | Abs, | |
|---|---|---|---|
| hours | 450 nm | 450 nm | Mean |
| 0 | 0 | 0 | 0 |
| 0.5 | 1.099 | 1.217 | 1.158 |
| 1 | 1.174 | 1.258 | 1.216 |
| 2 | 1.190 | 1.260 | 1.225 |
| 3 | 1.184 | 1.249 | 1.216 |
| 4 | 1.184 | 1.258 | 1.221 |
| 5.5 | 1.180 | 1.260 | 1.220 |

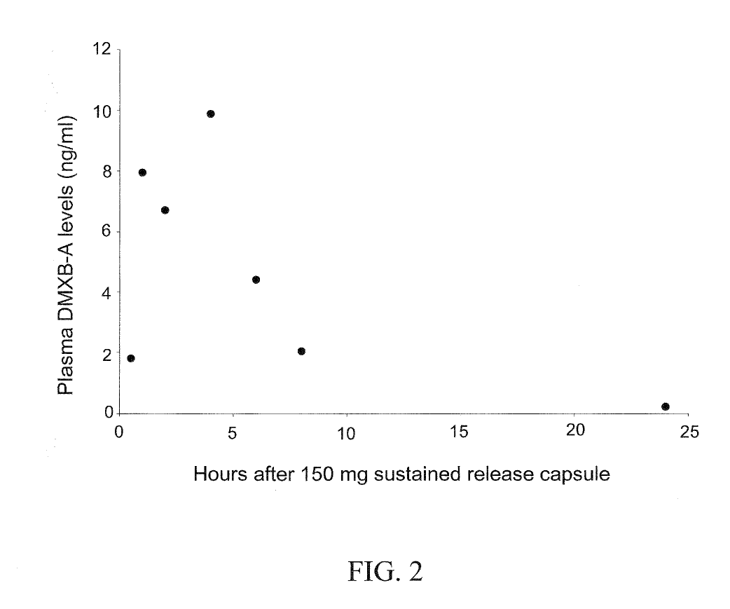

The same CR formulation and dosage of Table 1 was administered to 4 human volunteers with no adverse events experienced. The graph of FIG. 2 shows that the formulation is producing the sustained plasma levels of DMXB-A predicted from the in vitro tests. After one capsule, potentially therapeutic levels are seen for 6 to 8 hours. Averages of levels from two administrations in the same subject are shown in FIG. 2.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A controlled-release dosage form, comprising a therapeutically effective amount of an anabaseine compound and a controlled-release agent.

2. The controlled-release dosage form of claim 1, wherein said controlled-release agent is a controlled-release matrix and wherein said anabaseine compound is incorporated into the controlled-release matrix.

3. The controlled-release dosage form of claim 1, wherein said controlled-release matrix comprises a polymer.

4. The controlled-release dosage form of claim 3, wherein said polymer is nonionic.

5. The controlled-release dosage form of claim 3, wherein said polymer is water-soluble.

6. The controlled-release dosage form of claim 3, wherein said polymer is a gel-forming polymer.

7. The controlled-release dosage form of claim 3, wherein said polymer hydrates in an aqueous environment, forming a gelatinous (gel) layer on the outer surface of said dosage form, which retards wetting and disintegration of the interior of said dosage form, and wherein said gel layer swells or expands, increasing in thickness, as water penetrates into the dosage form and said anabaseine compound diffuses through said gel layer.

8. The controlled-release dosage form of claim 1, wherein said anabaseine compound is water-insoluble and is released from said dosage form primarily through erosion of said dosage form.

9. The controlled-release dosage form of claim 7, wherein said anabaseine compound is water-soluble and is released from said dosage form primarily through diffusion through said gel layer.

10. The controlled-release dosage form of claim 3, wherein said polymer is a cellulose polymer.

11. The controlled-release dosage form of claim 3, wherein said polymer is a cellulose ether.

12. The controlled-release dosage form of claim 3, wherein said polymer is hypromellose or methyl cellulose.

13. The controlled-release dosage form of claim 1, wherein said dosage form is a tablet or capsule.

14. The controlled-release dosage form of claim 1, wherein said anabaseine compound is an agonist of the alpha7 nicotinic acetylcholine receptor.

15. The controlled-release dosage form of claim 1, wherein said anabaseine compound is an arylidene-anabaseine.

16. The controlled-release dosage form of claim 1, wherein said anabaseine compound is DMXBA, or a pharmaceutically acceptable salt thereof.

17-19. (canceled)

20. A method for administering an anabaseine compound to a subject in a controlled-release fashion, comprising administering a controlled-release dosage form comprising a therapeutically effective amount of an anabaseine compound and a controlled-release agent.

21-22. (canceled)

23. A method for treating or preventing a disease or condition associated with a defect in, and/or malfunctioning of, a nicotinic acetylcholine receptor, the method comprising administering a controlled-release dosage form comprising a therapeutically effective amount of an anabaseine compound and a controlled-release agent.

24. The method of claim 23, wherein the nicotinic acetylcholine receptor is a neuronal nicotinic acetylcholine receptor of the brain, and the disease or disorder is Alzheimer's disease, schizophrenia, Parkinson's disease, or attention deficit-hyperactivity disorder; or wherein the nicotinic acetylcholine receptor is a non-neuronal acetylcholine receptor, and the disease or disorder is an inflammatory disorder, trauma, deficient angiogensis, excessive angiogenesis, or abnormal cell proliferation.

25-27. (canceled)

28. A method for preparing a controlled-release dosage form suitable for oral administration, the method comprising: a) blending one or more anabaseine compounds and a matrixing agent, and, optionally, one or more other ingredients to form a mixture; and b) forming the mixture into the controlled-release dosage form.

29-32. (canceled)
